(19) United States
(12) Patent Application Publication
(10) Pub. No.: US 2011/0245854 A1
(43) Pub. Date: Oct. 6, 2011

(54) DEVICES AND METHODS TO DELIVER, RETAIN AND REMOVE A SEPARATING DEVICE IN AN INTUSSUSCEPTED HOLLOW ORGAN

(75) Inventors: Darin Buxbaum, Palo Alto, CA (US); Charles Hsu, Upland, CA (US); Ivan Tzvetanov, San Francisco, CA (US); Fan Zhang, Menlo Park, CA (US); Michael Reo, Redwood City, CA (US)

(73) Assignee: HOURGLASS TECHNOLOGIES, INC., Redwood City, CA (US)

(21) Appl. No.: 13/111,876

(22) Filed: May 19, 2011

Related U.S. Application Data
(63) Continuation-in-part of application No. 11/870,096, filed on Oct. 10, 2007.
(60) Provisional application No. 61/347,089, filed on May 21, 2010.

(51) Int. Cl. A61B 17/00

(52) U.S. Cl. 606/153

(57) ABSTRACT

The present invention is an improved separating device for providing the malabsorptive component of a minimally invasive weight reduction system. This device may be a sleeve, liner, or tubular sheath that alters absorption of compositions through walls of hollow organs (stomach, intestines). The improvement involves better long-term retention through the ability of the device to engage with the interserosal fibrotic tissue formed in an intussusception. The separating device may be a distal extension of a retaining member that is directly engaged with and retained by intussuscepted tissue. Intussusception shrinks a hollow organ to provide a volume reducing restrictive component of a weight reduction system. Securing the separating device to an intussusception avoids retention problems of reference art devices as interserosal fibrosis helps secure the device. Also provided are an apparatus for retaining the device, a device and method for delivering the separating device, and a method for removing it.

### CROSS-REFERENCES TO RELATED APPLICATIONS

This application claims priority as a nonprovisional application from U.S. Provisional Application No. 61/347,089, filed May 21, 2010 incorporated herein by reference in its entirety. This application is a continuation-in-part (CIP) of co-pending, commonly owned U.S. application Ser. No. 11/870,096, filed Oct. 10, 2007 incorporated herein by reference in its entirety. This application may also be related to U.S. application Ser. No. 12/265,509, filed Nov. 5, 2008; and U.S. application Ser. No. 12/265,539, filed Nov. 5, 2008; both of which are also herein incorporated by reference in their entirety.

### BACKGROUND

1. Field of Invention

The present invention relates to improving the delivery, retention, and removal of separating devices implanted in hollow organs for the restrictive and malabsorptive functions they provide. More specifically, the invention improves long-term retention of separating devices, including gastrointestinal sleeves, by engaging them with tissue that has been altered to provide a restrictive function. Most specifically, the invention makes use of the interserosal fibrotic tissue created through formation of intussusceptions to retain conventional and improved separating devices in the gastrointestinal tract to provide short and long-term weight loss following minimally invasive bariatric surgery.

2. Description of the Related Art

Obesity has become a worldwide health epidemic. Estimates by the World Health Organization suggest that if the increasing incidence of obesity continues then by the year 2015 approximately 1.5 billion people will be affected. Bariatric surgery (or surgical treatment of obesity) has emerged as superior to behavioral and pharmacological treatment methods for significant and sustainable weight loss in affected individuals. The number of bariatric surgeries performed in the United States has increased 9 fold in the past decade. Outside of the United States, the number of bariatric surgeries performed in that same period has more than tripled. (See “Surgical treatment of obesity” by Marc Ward, MD and Vivek Prachand, MD, FACS in Gastrointestinal Endoscopy, Vol. 70, No. 5 (2009) pp. 985-990.)

Modern bariatric surgical procedures aim to induce weight loss in two main ways: (i) restriction, which means volumetric reduction of the gastrointestinal (GI) organs to decrease the volume of food and the amount of calories a patient can comfortably consume at a given time; and (ii) malabsorption, which limits energy absorption by effectively bypassing portions of the GI tract through which food is absorbed (and nutrients sequestered). Portions of the GI tract can be effectively bypassed with a separating device that creates a barrier to prevent secretions (from the GI tract walls) that breakdown food from entering the device to access food. Substantially undigested, residual food that cannot be broken down due to reduced exposure to digestive secretions and enzymes simply passes through the remainder of the tract until it is excreted. Two procedures that rely primarily upon the former principle, restriction, are the laparoscopic adjustable gastric band (LAGB) and the vertical sleeve gastrectomy (SG). Two procedures that rely primarily upon the latter principle, malabsorption, are Roux-en-Y gastric bypass (RYGB) and biliopancreatic diversion with or without duodenal switch (BPD and BPD/DS).

While bariatric surgical procedures appear to offer more promise for seriously obese patients than pharmaceutical approaches and behavioral modification, there are nonetheless shortcomings with currently available options. The laparoscopic adjustable gastric band (LAGB) involves embracing the upper portion of the stomach with a circular band to form a pouch. The band can be tightened and released (adjusted) to decrease and increase, respectively, the volume of the pouch and to control a rate of emptying from the pouch into the remainder of the stomach. While generally effective for moderately obese patients, the band has much lower rates of success in more seriously obese individuals. Additionally, the weight loss following band implantation is typically gradual which can be discouraging for some patients. Complications include mild upper GI symptoms, band slippage, prolapse, and more rarely erosion into the gastric lining and esophageal dilation.

Vertical sleeve gastrectomy (SG) is more invasive in that it involves excising much of the upper curvature or fundus portion of the stomach to leave behind a narrower, tubular shaped organ. The resected stomach resembles an elongated sleeve rather than a rounded body. Problems include risk of vitamin B₁₂ deficiency and gastric leaks near the staple line at the gastroesophageal (GE) junction.

Roux-en-Y gastric bypass (RYGB) involves both restrictive and malabsorption aspects. First, a pouch is created in the upper portion of the stomach. Then, the jejunum (middle portion of the small intestine between the duodenum proximally and the ileum distally) is divided into two limbs and one of the limbs is anastomosed to the pouch. These structural modifications result in bypassing the release of food-stimulated gut hormones and absorption of food in the duodenum and proximal jejunum. Drawbacks of the procedure are that it takes longer and is more difficult compared to other contemporary procedures and there is a higher frequency of perioperative complications. Other downfalls include a likelihood of vitamin B₁₂ and iron deficiency, and a strong association between RYGB and dumping syndrome (which may also be a benefit due to its deterrent effect on consumption of highly processed carbohydrates).

Biliopancreatic diversion with or without duodenal switch (BPD and BPD/DS) involves sleeve gastrectomy (SG) followed by transaction of the duodenum (upper portion of the small intestine) distal to the pylorus (lower portion of the stomach with a ring of smooth muscle, or sphincter, that controls emptying into the duodenum). For BPD/DS the ileum (lower portion of the small intestine) is typically divided proximal to the ileocecal junction and the distal limb of ileum is then anastomosed to the postpyloric duodenum with a distal anastomosis of the distal biliopancreatic limb proximal to the ileocecal junction. BPD/DS is more effective than other methods in treating super-obese patients. Nutritional deficiencies are a common negative side effect, including calcium malabsorption and an increase in parathyroid hormone, hypoalbuminemia, and fat soluble vitamin deficiencies (including vitamin A, vitamin K, and vitamin D).

The present invention seeks to provide a device and procedure that combines the restrictive and malabsorptive functions of the above procedures in a less invasive manner with a significantly improved effectiveness to invasiveness ratio that provides both immediate and continuing weight loss and facilitates long-term weight maintenance. Reduced invasiveness means less risk of perioperative complications and a lower mortality rate. Additionally, patients not healthy enough for traditional surgery may nevertheless be candidates for non-traditional minimally invasive procedures.

The present invention seeks to provide an alternative superior to reference art devices and methods for delivering, retaining, and removing separating devices in the gastrointestinal system. Setbacks of reference art devices and methods, including poor retention, are overcome in part by providing an improved anatomic platform of intussuscepted interserosal fibrotic tissue upon/against which to hold the separation device, as disclosed further herein.

It would be desirable to provide a device for weight loss that can be delivered with minimal invasiveness and remains in the same desired position over time. It would also be desirable to provide a device for weight loss that does not cut into linings of the GI tract and hollow organs therein. It would further be desirable to provide a device that does not detrimentally impair absorption of vitamins and nutrients. It would additionally be desirable to provide a device that induces prompt weight loss, maintainable over time, without requiring incising and reconstructing major portions of the stomach and intestines. Finally, it would be desirable to provide a weight loss device that does not require significant time off work and apart from normal daily activities for the implantation procedure.

The present invention addresses these problems and meets these and other needs.

### SUMMARY OF THE INVENTION

Tissue can be altered to reduce the volume of a hollow organ or provide a restrictive function, such as by an intussusception. The terms “intussuscept,” “intussusception,” “intussuscepting,” “intussuscepted,” and the like refer to a geometry created by telescoping one part of a hollow organ onto or into another part of the hollow organ.

When an intussusception is formed, the tissue can be stabilized in that configuration through the use of temporary or permanent retaining members, including anchors, sutures, elastic retaining bands, glue, or cauterization or electrosurgery tools that seal or weld the tissue portions together. Serosal surfaces that become apposed in this configuration can then create interserosal fibrosis as part of the healing response. Various tissue growth-enhancing compositions and additives may, but need not, be added to the joined tissue portions to expedite and exacerbate this healing process to assist in the development and proliferation of interserosal fibrotic tissue. Also, by creating an intussusception, the volume of a hollow organ is reduced. Volume reduction limits the amount of food or other materials an organ can hold and reduces tolerance to excessive amounts, thereby inhibiting the ability or desire to consume. A preferred application of this process is in the stomach to reduce the amount of food it can hold before a person experiences discomfort and biological cues to stop eating. Forming one or more intussusceptions in stomach tissue makes it easier for the stomach to fill up and may increase gut hormones related to satiety (e.g. GLP-1 and peptide YY) and/or reduce blood ghrelin levels associated with early-phase intake.

In addition to volumetric reduction of hollow organs, another way to limit the amount of calories or other materials they effectively receive is to reduce processing of materials in the organs. Processing of materials typically occurs as enzymes and vitamins from the walls of an organ are secreted to the inside of the organ to digest or transform the materials. Further processing may occur as partially digested, transformed, or dissolved materials pass outward through the walls of an organ into the blood stream where more processing occurs at the cellular level along with distribution throughout the body. Medical devices that block the flow of digestive enzymes (or other compositions and fluids) from the outer walls of an organ to the hollow inside, and from the hollow inside of an organ outward through the walls, can be used to inhibit or limit processing and reduce absorption. Food and other materials that are not properly processed and absorbed will eventually be excreted.

The present application introduces the invention of integrating restriction (volume reduction) and malabsorptive (barrier layer) components for better long-term and short-term results and improved delivery, retention, and removal of the malabsorptive component. The naturally strong interserosal fibrotic tissue that forms with an intussusception provides a superior platform from which to retain a malabsorption sleeve.

When used in the stomach and intestines, the malabsorption sleeve has multiple functions, including providing: i) a reduced tubular cavity or canal limiting the amount of food that can be received in the hollow organ(s), and ii) a protective slide that expedites movement of food through the GI system while reducing or inhibiting the natural process of digestion and absorption. To achieve the first function, the sleeve can have a smaller diameter than the diameter of the hollow organ in which it is implanted. To achieve the second function, the sleeve can have an inner lining that repels food contents and walls that selectively restrict entry of digestive enzymes and exit of broken down food.

According to the devices and methods described herein the malabsorption sleeve may be engaged with the tissue intussusception in a number of ways. According to one embodiment, the proximal end of the sleeve may be folded into the intussusception at the time the intussusception is formed. By enabling sleeve implantation and tissue intussusception in a single step the present invention could greatly reduce procedure time. If the proximal end of the sleeve is semi-porous it may permit tissue to grow or seal through it, or adhesives to bond through it. According to another embodiment, the proximal end of the sleeve can be retained to an outer wall of the intussusception, especially a mucosal surface, rather than sandwiched between the tissue that forms the intussusception. This may occur through a single-step or multi-step procedure in which a proximal end of the sleeve is retained to the intussusception at the time it is formed or attached after.

The devices and methods described herein may be especially useful for delivering, retaining, and removing a separating device within an intussuscepted hollow organ in the body. Separating devices provide a barrier layer that serves to delay, prevent, or reduce mixing of biological fluids on the two sides of the device.

According to one of several aspects of the present invention a method for retaining a malabsorptive component of a weight reduction system to a restrictive component of a weight reduction system is provided, which involves introducing the restrictive component in a hollow organ, introducing the malabsorptive component in the hollow organ, and attaching the malabsorptive component to the restrictive component. The restrictive component may be introduced in a hollow organ by either inserting it in a hollow organ or forming it in a hollow organ. For example, the restrictive component may be inserted in a hollow organ when it takes the form of a space-occupying or volume-occupying object that reduces stomach volume available for food. Insertion is preferably minimally invasive and transoral, for example delivery of inflatable space-occupying objects may be accomplished in this manner as they can be inserted transorally in a deflated state. As another example, the restrictive component may be formed in a hollow organ when it involves telescoping a first region of tissue into a second region of tissue resulting in an intussusception. The malabsorptive component may also be introduced in a hollow organ by either inserting it in a hollow organ or forming it in a hollow organ. For example, the malabsorptive component may be inserted in a hollow organ when it takes the form of an impermeable or semi-permeable separating device or sleeve which impedes flow of food and/or digestive enzymes across it. Insertion of a separating device or sleeve is preferably minimally invasive and transoral. As another example, the malabsorptive component may be formed in a hollow organ when it involves reconfiguring the shape, entry/exit points, or surface texture of one or more hollow organs to impede flow of food and/or digestive enzymes across a hollow organ to reduce absorbed calories. Attaching the malabsorptive component to the restrictive component may include, but is not limited to, attaching a separating device to an intussuception or hanging a sleeve from an intussusception as the intussusception makes a superior platform for retention of the malabsorptive component (separating device or sleeve).

According to another aspect of the present invention a method for retaining a separating device in a region of an intussusception is provided, which involves retaining the separating device in the region of the intussusception, in which the intussusception is formed by telescoping a first region of stomach tissue into a second region of stomach tissue. Retaining may involve attaching a proximal end of the separating device into the intussusception at the time the intussusception is formed. Retaining may involve at least one element selected from the group consisting of: inserting mechanical elements through the intussusception, enlarging a proximal portion of the separating device, delivering energy, activating an adhesive, and applying an adhesive.

According to another aspect of the present invention an apparatus for integrating a malabsorptive component and a restrictive component of a weight reduction system is provided, including a self-retaining separating device configured for attachment to an intussusception. The separating device may be deployable to self-retain by the separating device hanging from a stoma defined by the intussusception. The proximal end of the separating device may be enlargeable to the extent that it is larger than a stoma defined by the intussusception. The proximal end of the separating device may be elastic thereby enabling the proximal end to wrap around an outer surface of the intussusception. The proximal end of the separating device may be configured to bias outwardly. The separating device may have at least two states, an insertion/removal state and a retention state. The at least two states may be restricted to a proximal end of the separating device. The separating device may have an elastic portion that exerts a force (or forces) inwardly resulting in the retention state. The separating device may include retractable elements that exert a force (or forces) inwardly resulting in the retention state. The separating device may have a biasing portion that exerts a force (or forces) outwardly resulting in the retention state. The separating device may include extendable elements that exert a force (or forces) outwardly resulting in the retention state. A proximal portion of the separating device may have a surface structure capable of activation to engage with tissue adjacent the intussusception.

According to another aspect of the present invention an apparatus for integrating a malabsorptive component and a restrictive component of a weight reduction system is provided, including a separating device and a retaining member configured to secure the separating device to an intussusception. The retaining member may include any combination of tissue-penetrating elements, an adhesive, or a material configured to respond to energy application from an electrosurgical or electrocautery instrument. The apparatus may also include a means for dispersing pressure created by the retaining member. The means for dispersing pressure may be an intrastomal device, an extrastomal device, both an intrastomal device and an extrastomal device, balloons, springs, retractors, fluids, holes, channels, grooves, surface texture, variable thickness, variable materials, and the like. A distal portion of the retaining member may define the separating device. At least a portion of the retaining member may be configured to deploy. The retaining member may be elastic for engagement with an outer surface of a lumen defined by walls of the intussusception thereby securing the retaining member about the lumen.

According to another aspect of the present invention a separating device configured to be retained in a hollow organ, for altering absorption of one or more compositions through one or more walls of one or more hollow organs is provided, including an elongated tubular body enclosing a pathway along which one or more compositions can travel until reaching a distal end of the separating device and a means for retaining a proximal end of the separating device in a region of an intussusception. The means for retaining the proximal end of the device in a region of the intussusception may include interaction between a mucosal surface of an organ and the device. The means for retaining the proximal end of the device in a region of the intussusception may include fitting the device between tissue on one or more outside walls of the proximal end of the device. The means for retaining the proximal end of the device in a region of the intussusception may include engaging tissue that is or becomes interserosal fibrotic tissue. The means for retaining the proximal end of the device in a region of the intussusception may include engaging opposed tissue. The means for retaining the proximal end of the device in a region of the intussusception may include engaging an area of interserosal apposition formed by an intussusception. The means for retaining the proximal end of the device in a region of the intussusception may include a friction fit. The friction fit may involve the proximal end of the separating device wrapped around the intussusception so as to exert a force (or forces) inward. The friction fit may involve the proximal end of the separating device inserted within the intussusception and exerting a force (or forces) outward. The proximal end of the separating device may be elastic. The proximal end of the separating device may be disposed to bias outwardly, or “springy”. The proximal end of the separating device may be expandable. The means for retaining the proximal end of the separating device in a region of the intussusception may include one or more anchors. The separating device may be a distal part of a retaining member on an inside of a lumen formed by walls of the intussusception. The separating device may be a distal part of a retaining member on an outside of a lumen formed by walls of the intussusception. The means for retaining the proximal end of the separating device in a region of the intussusception may include retaining the proximal end of the separating device to a retaining member configured to fit inside the intussusception. The means for retaining the proximal end of the separating device in a region of the intussusception may include retaining the proximal end of the separating device to a retaining member configured to fit outside the intussusception. The proximal end of the separating device may be retained directly by the retaining member. The proximal end of the device may be retained indirectly by the retaining member through an intermediate connector. The means for retaining the proximal end of the separating device in a region of the intussusception may include a proximal end of the separating device that is enlargeable or enlarged such that it is larger than a stoma formed by the intussusception. The separating device may also include a means for dispersing pressure created by the means for retaining.

According to another aspect of the present invention an apparatus for retaining a device in a region of an intussusception formed in a hollow organ is provided, including one or more of a retaining member configured to fit inside walls formed by the intussusception and/or a retaining member configured to fit outside walls formed by the intussusception.

According to another aspect of the present invention a delivery device for implanting an apparatus as disclosed above in a hollow organ is provided, including an elongated shaft and a distal element that is both collapsible and deployable at a distal end of the shaft. The collapseable/deployable distal element is configured to be atraumatically introduced into the hollow organ in a collapsed configuration and deployment of the distal element causes the separating device to extend from a retracted configuration. The collapsible/deployable distal element may be umbrella-shaped.

According to another aspect of the present invention a method for delivering a separating device to a hollow organ is provided, which involves: inserting a delivery device into the hollow organ, with the separating device retained thereto in a retracted configuration; reconfiguring tissue around the delivery device thereby forming an intussusception; securing the separating device in a region of the intussusception, thereby using the intussusception to retain the separating device; removing the delivery device; and deploying the separating device from a retracted to a deployed configuration.

According to another aspect of the present invention an apparatus for removing a separating device, or any portion thereof, from an intussusception, is provided including a housing, a folding arm that fits within the housing, and a cutter at a distal end of the folding arm. The folding arm is configured to telescopically extend from the housing, swivel between an axis parallel to a shaft of the housing and an axis perpendicular to the shaft of the housing, and rotate around the axis parallel to the shaft of the housing.

According to another aspect of the present invention a method for removing all or a portion of a separating device from a hollow organ is provided, which includes: inserting a housing into the hollow organ, the housing comprising a folding arm therein with a cutter at a distal end of the folding arm, the folding arm configured to swivel between an axis parallel to a shaft of the housing and an axis perpendicular to the shaft of the housing; telescopically extending the folding arm from the housing along the axis of the shaft of the housing; swiveling the folding arm outward away from the shaft of the housing in a direction such that the folding arm approaches the axis perpendicular to the shaft of the housing; engaging the cutter on the distal end of the folding arm with the separating device; and rotating the folding arm around the separating device such that the cutter at the distal end moves through the perimeter of the separating device, thereby cutting off at least a portion of the separating device.

According to another aspect of the present invention a method for removing all or a segment of a separating device from an intussusception in a hollow organ is provided, which involves: cutting a suture that engages the separating device; grasping an end of the cut suture; and pulling while grasping to remove the suture, thereby detaching the separating device from the intussusception or detaching a segment of the separating device from another segment of the separating device.

These and other aspects and advantages of the invention will become apparent from the following detailed description, and the accompanying drawings which illustrate, by way of example, the features of the invention.

### BRIEF DESCRIPTION OF THE DRAWINGS

FIG. 1 is a schematic view depicting a hollow organ within which an intussusception may be formed to retain a separating device in a region thereof.

FIG. 2 is a schematic view depicting a separating device, e.g. a GI sleeve, retained in a region of an intussusception by wrapping around outer walls of the intussusception and through use of mechanical retaining elements (anchors, staples, sutures, pins, etc.).

FIG. 3 is a schematic view depicting a separating device as in FIG. 2 and further comprising an additional retaining and/or pressure-dispersing element (e.g. an intrastomal device) that fits inside the walls of the intussusception (within the stoma formed by intussuscepted tissue), with the mechanical retaining elements extending through the outer sleeve, the intussuscepted tissue, and the intrastomal device.

FIG. 4 is a schematic view depicting a separating device as in FIG. 3 and further comprising an additional retaining and/or pressure-dispersing element (e.g. an extrastomal device) that fits outside the walls of the intussusception (outside and around the stoma formed by intussuscepted tissue) with the separating sleeve being wrapped around outside the extrastomal device. The mechanical retaining elements extend through the sleeve, the extrastomal device, the intussuscepted tissue, and the intrastomal device.

FIG. 5 is a schematic view depicting a separating device substantially as in FIG. 4 but with the mechanical retaining elements extending only through the extrastomal device, the intussuscepted tissue, and the intrastomal device but not the sleeve. The sleeve may be friction fit to wrap-around and hug the outside of the extrasomal device.

FIG. 6 is a schematic view depicting a separating device substantially as in FIG. 3 but with the proximal end of the sleeve at the distal end of the intussuscepted tissue such that the sleeve is initially deployed and extended upwards (proximally) and then wraps around (e.g. upon reaching upper walls of the hollow organ) to reverse direction and extend distally.

FIG. 7 is a schematic view depicting a separating device as a distal extension of a retaining and/or pressure-dispersing element (e.g. an intrastomal device) that fits inside the walls of the intussusception (within the stoma formed by intussuscepted tissue). Mechanical retaining elements extend through the extrastomal device, the intussuscepted tissue, and the intrastomal device.

FIG. 8 is a schematic view depicting a separating device wrapped around a retaining and/or pressure-dispersing element (e.g. an intrastomal device) that fits inside the walls of the intussusception (within the stoma formed by intussuscepted tissue). This embodiment is comparable to that shown in FIG. 4 but with the separating device inside the intussusception wrapped around the intrastomal device rather than outside the intussusception wrapped around the extrastomal device. Mechanical retaining elements extend through the extrastomal device, the intussuscepted tissue, the sleeve, and the intrastomal device.

FIG. 9 is a schematic view depicting a separating device fit inside and extending through a retaining and/or pressure-dispersing element (e.g. an intrastomal device) that fits inside the walls of the intussusception (within the stoma formed by intussuscepted tissue). As an alternative to using mechanical retaining elements (e.g. anchors) or a friction fit (e.g. elastic band) that exerts tension inward to hold the separating device in a region of the intussusception, the proximal end of the separating device is enlarged and lodged into a hollow proximal cavity formed above the intussuscepted tissue such that it will not fit through the stoma created by the intussusception. Also present is an extrastomal device.

FIG. 10 is a schematic view depicting a separating device and retention apparatus as in FIG. 9, further comprising mechanical retaining elements (e.g. anchors, pins, etc.) that supplement or complement the enlarged proximal end of the separating device to retain it in a region of the intussusception. The proximal anchors traverse the walls of the hollow organ, the intussuscepted tissue, the walls of the proximal cavity, and the proximal end of the separating device. The more distal anchors traverse the extrastomal device, the intussuscepted tissue, and the intrastomal device (and optionally, the elongated section of the separating device).

FIG. 11 is a schematic view depicting a separating device substantially as in FIG. 7 except that the sleeve is hung from a distal portion of the intussuscepted tissue using anchors or connector elements (e.g. circular rings as shown) rather than being a distal extension of the intrastomal device.

FIG. 12 is a schematic view depicting a separating device as in FIG. 11 with the mechanical retaining elements (anchors, connectors, etc.) used to hang the separating device also extending through the extrastomal device in addition to the distal portion of the intussuscepted tissue.

FIG. 13 is a schematic view depicting a device and method for delivering the separating device and retention apparatus as disclosed herein, in which the separating device is an extension of the extrastomal device, e.g. outer mesh or silicone bands of the retention apparatus.

FIG. 14 is a schematic view depicting a device and method for delivering the separating device and retention apparatus as disclosed herein, in which the separating device is an extension of the intrastomal device or inner mesh of the retention apparatus.

FIG. 15 is a schematic view depicting exemplary unfurling elements which may be used to deploy the separating device from a retracted (e.g. rolled up) to an extended (e.g. unrolled) configuration after delivery.

FIG. 16 is a schematic view depicting an example of a ring-shaped unfurling element that wraps around distal ends of the separating device.

FIG. 17 is a schematic view depicting one example of a device and method for removing the separating device or a portion thereof, in which the device comprises a telescopically extendable folding arm with a cutter on its distal end configured to swivel and rotate around the central axis to cut off the sleeve.

FIG. 18 is a schematic view depicting another example of a device and method for removing the separating device or a portion thereof, in which endoscopic scissors and/or forceps are used to cut, grasp, and pull a running suture to detach the sleeve.

FIG. 19 is a schematic view depicting another example of a device and method for removing the separating device, according to the embodiment in which the device has an enlarged proximal portion, in which a grasping tool is used to grasp and remove the enlarged proximal end of the sleeve from within the hollow cavity.

FIG. 20 is a schematic view of a stable stomach bypass with partial gastric fluid exposure showing a bypass liner originating at an intussusception and including segments of various lengths, some of which may allow gastric fluid passage for partial digestion.

FIG. 21 is another schematic view of a stable stomach bypass with partial gastric fluid exposure showing a distal end of the bypass liner secured by intussuscepting the small intestine either above or below outflow from the gallbladder or pancreas and also showing a mesh at the outflow tract of the pancreas.

FIG. 22 is another schematic view of a stable stomach bypass with partial gastric fluid exposure showing a distal end of the bypass liner secured by a balloon.

FIG. 23 provides a detailed depiction of a distal portion of a device used for forming an intussusception having a collapsed configuration.

FIG. 24 provides a detailed depictions of a distal portion of a device used for forming an intussusception having an expanded configuration.

### DETAILED DESCRIPTION OF THE PREFERRED EMBODIMENTS

In its several embodiments, the present invention provides i) an improved separating device for implantation within a gastrointestinal (GI) tract of a patient to facilitate weight loss; ii) an apparatus for retaining the separating device; iii) a delivery device for implanting the separating device; iv) a removal device for retrieving the separating device (or a portion thereof); v) a method of delivering the separating device; vi) a method of retaining the separating device; and vii) a method of retrieving the separating device.

### Definitions

As used herein, the following terms are defined as indicated below, consistent with the principle that a patentee can be his own lexicographer.

The terms “hollow organs” include, but are not limited to, the organs of the gastrointestinal tract including the esophagus, the stomach, and the small intestines.

The terms “intussuscept,” “intussusception,” “intussuscepting,” “intussuscepted,” and the like refer to the geometry created by telescoping one part of a hollow organ onto or into another part of the hollow organ.

The phrase “in a region of an intussusception” refers to a location at or within a certain proximity to an intussusception formed in a hollow organ. As used herein, this proximity may be 1 cm or less, 2 cm or less, 3 cm or less, 4 cm or less, 5 cm or less, 6 cm or less, 7 cm or less, 8 cm or less, 9 cm or less, or 10 cm or less depending upon i) the particular hollow organ, ii) the specific medical application or purpose, and iii) the anatomy of a particular individual. Preferably, the proximity referred to by “in a region of an intussusception” is 0 to 3 cm from an intussusception.

The phrase “means for dispersing pressure” refers to any elements that disperse pressure in a region of an intussusception. For example, retention of the separating device in the region of the intussuception may result from a self-retaining separating device exerting one or more forces inwardly or outwardly, against, around, or adjacent to the intussusception. Such forces may result in pressure changes. A means for dispersing pressure may be relied upon to release and redistribute the pressure(s) resulting from retaining forces in order to restore the anatomy to mimic a natural biomechanical state with all pressures at acceptable levels. The phrase includes intrastomal and/or extrastomal devices that fit inside or outside walls formed by the intussuscepted tissue. However, other elements for dispersing pressure are also contemplated. These may include balloons, springs, retractors, fluids, holes, channels, grooves, surface texture, variable thickness, variable materials, etc. Pressure buildup may, but need not, be created by retaining members that function to hold the separating device in a region of the intussusception. Means for dispersing pressure may be provided to alleviate any unwanted pressure buildup created by the retaining members. Some types of retaining members may have an inherent pressure-dispersing function.

The term “restrictive component” is a broad term that may refer to tissue being reconfigured from its previous or natural configuration which may or may not actually restrict or decrease the volume of a hollow organ that includes the tissue. For example, an intussusception as a restrictive component may reconfigure tissue to restrict passageways in certain regions without decreasing volume or it may simply reconfigure tissue without even restricting passageways. In some, but not all, situations an intussusception may be used to restrict the volume of a hollow organ but this is not necessary or implied by the term “restrictive component”. Restrictive component may also refer to a volume-occupying object that takes up space in a hollow organ and restricts the remaining volume available for food without reconfiguring any tissue.

The terms “retaining member(s)” or “means for retaining” (in reference to retaining a separating device in position within or against intussuscepted tissue of a hollow organ) refer broadly to any feasible and biocompatible means for retaining. These means may include techniques that transform the protein structure of tissue and cause it to bond with itself such as tissue welding, cauterizing, and electrosurgery. These means may include sticky compositions such as resins, adhesives, glues, cements, and tissue-mimicking materials. These means may include elastic bands exerting tension inward (when stretched) that cause the separating device to friction fit with the intussusception directly or to an intermediate device (intrastomal device, extrastomal device, inner mesh, outer mesh) that itself retains the separating device near the intussusception. Accordingly, intrastomal and extrastomal devices are also considered as retaining members and means for retaining. These means for retaining may also include springs or inflatable elements exerting outward forces that cause the separating device to expand outwardly against walls of a hollow organ (e.g. at the gastroesophageal junction) thereby friction fitting itself within a lumen of the GI tract. These means may also include traditional physical means for securing structures in the anatomy including anchors, staples, pins, clips, and sutures. The aforementioned examples are intended to be illustrative rather than exhaustive or limiting.

The terms “separating device” mean a device that divides two or more regions of a hollow organ to form a barrier between the regions. The barrier may be permanent or temporary (bioabsorbable, biodegradable, or bioerodible dissolving over time) and may also be complete, blocking all elements from crossing it, or selective (semi-permeable). The barrier may function to block transfer of materials in one direction or in both directions (inside to outside, outside to inside), or block some materials from moving in one direction and other materials from moving in a different direction. For example, block enzymes from coming in and block food from going out. The barrier changes the ability of materials to move freely throughout the hollow organ and to pass from one side of the hollow organ to another side (e.g. inside the cavity to outside into the bloodstream or outside from the walls to inside the cavity). Such a separating device may be a tubular sheath, a sleeve, or a liner with substantially open ends and impermeable or semi-permeable walls that inhibit or selectively inhibit passage of materials in/out. In this manner, the separating device thereby influences breakdown and absorption of food materials inside it within hollow organs of the GI tract.

Using the interserosal fibrotic tissue formed by one or more intussusceptions to retain the separating device improves long-term device stability and retention. The separating device can engage directly with this tissue, before, during, or after expected fibrosis formation, or indirectly by an intermediate element connecting the separating device and the intussusception or the interserosal fibrotic tissue.

Of note, all descriptions of utilization of interserosal fibrosis for retention of the separating device refer to interserosal fibrosis which may be pre-existing, may still be forming, or may not yet be formed but is reasonably expected to form as a result of the intussuscepted geometry.

Without penetrating the wall of a hollow organ the separating device will be adjacent to the inside mucosal surface of a hollow organ. However, the means for retaining can nevertheless engage or interact with the strong and supportive interserosal fibrotic tissue through penetration, although penetration is not essential as alternative techniques for engaging the interserosal fibrotic tissue to hold the separating device are available. For example, it is possible to friction fit the separating device to the intussusception with a non-penetrating or minimally penetrating retaining means. These include: (i) an elastic band at the proximal end of the separating device, placed outside of the intussusception closing in upon it, or (ii) a springy or expanding/swelling band at the proximal end of the separating device, placed inside a lumen of the intussusception pushing out against it. The inwardly directed elastic band or the outwardly directed springy or expanding/swelling bands may compress the device thereby retaining it. Alternatively, when tissue-penetrating elements, such as anchors, staples, or sutures, are used they physically extend into the interserosal fibrotic tissue. Preferably, tissue-penetrating retaining members enter interserosal fibrotic tissue of the intussusception through the mucosal surface of a hollow organ wall. Another alternative is for electrosurgical, electrocautery, or other energy-delivering surgical tools to weld or seal the proximal end of the separating device to the intussusception. Energy-delivery techniques may result in modifying the structure of the interserosal fibrotic tissue or other surrounding tissue.

The proximal end of the separating device may comprise one or more elements that are in themselves the means for retaining or that work together with other elements to become or assist the means for retaining. For example, when a friction fit is used to secure the separating device around the outside or the inside of the intussusception, the proximal end of the separating device may comprise an elastic band (that hugs inwardly) or a springy band (that exerts pressure outwardly) built into it so that a separate step of inserting and attaching this component is not required. When energy-delivering surgical tools are used to weld the separating device to the intussusception, the proximal end of the device may be designed to perform appropriately upon energy application to bond well with the intussuscepted tissue or intermediate retaining elements (meshes, adhesives, stents, etc.) on the surface thereof. For example, the proximal end of the device may be made of or patterned with a material that forms an adhesive that bonds well with tissue upon application of energy from electrosurgical tools.

Tissue-penetrating elements, including anchors, staples, and sutures, may be less suited for incorporation directly within the proximal end of the separating device. However, in some embodiments, if the tissue-penetrating elements are housed or shielded appropriately so as not to compromise an atraumatic introduction of the separating device they too can be integrated. Including the means for retaining, or portions of it, in the proximal end of the separating device eliminates extra surgical steps and reduces procedure time as more elements are delivered at the same time and do not have to be separately inserted and positioned.

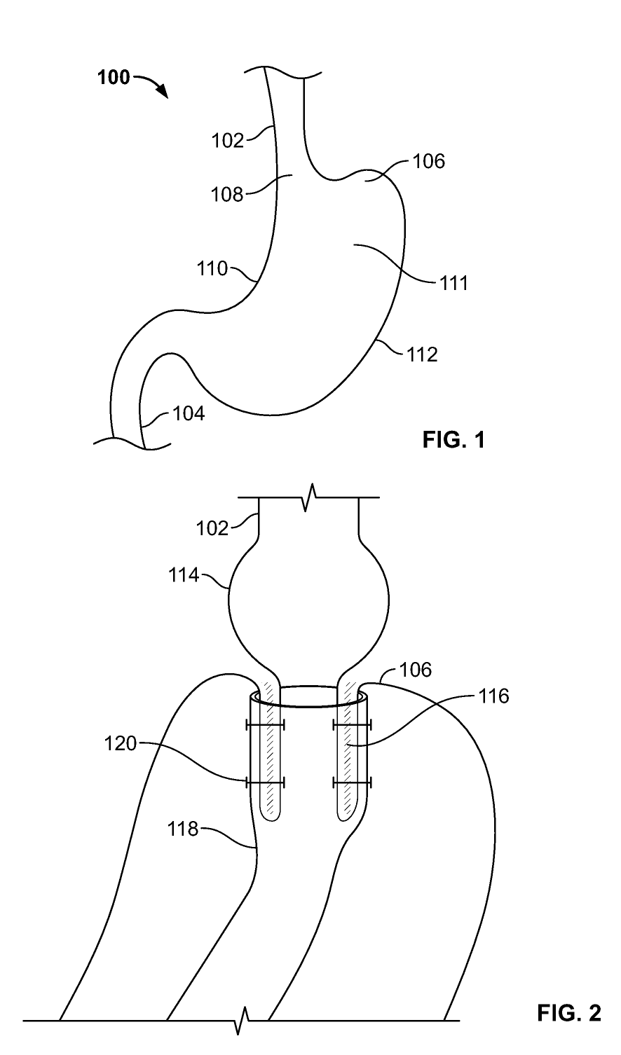

FIG. 1 shows a simplified depiction of a portion of the gastrointestinal (GI) tract 100 with the esophagus 102 entering a hollow organ 111, here the stomach, at the gastroesophageal (GE) junction 108. The stomach is shaped to have an inner curvature 110 and outer curvature 112 and the upper right portion of the stomach is called the fundus 106. Also shown is the upper portion of the small intestines, or duodenum, 104. The area distal to the gastroesophageal junction region is a preferred site for attachment and retention of a proximal end of a separating device. The separating device should extend distally into the duodenum 104.

FIG. 2 shows a simplified depiction of a tubular separating device 118 which is anchored to both the organ walls and the circumferential tissue which exists outside of the gastric walls. In general, the methods described here are used to retain a separating device 118 within a hollow organ 111 by utilizing an intussuscepted geometry. As shown in FIG. 2, the tubular separating device 118 is being retained within the organ by placement over the intussuscepted tissue 116, with tissue anchors 120 which traverse the intussuscepted tissue 116 as well as the extraluminal tissue. The tissue anchors 120 may be geometrically distributed in one or more rows in order to increase the robustness of device retention. The device is illustratively shown in an upper region of a stomach although alternative locations in the GI tract (other parts of the stomach, in the esophagus, in the intestines, etc.) are also contemplated as within the scope of the present invention. Generally, formation of an intussusception creates a proximal cavity 114 above the intussuscepted tissue.

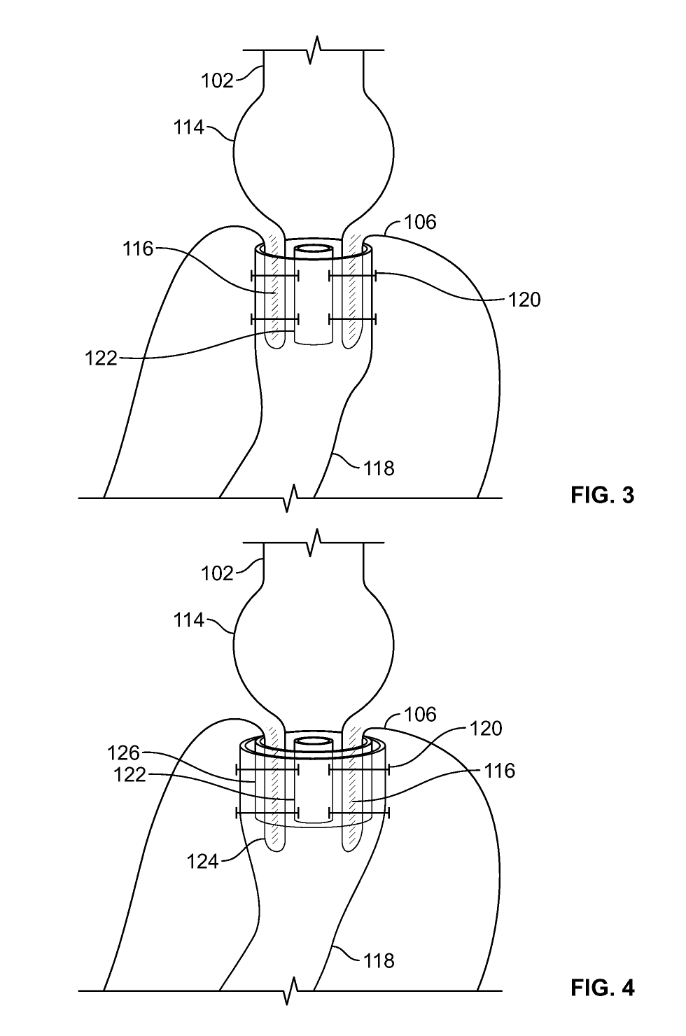

FIG. 3 shows another embodiment in which the separating device 118 is positioned over or outside of the intussuscepted tissue 116, and the tissue anchors 120 traverse an additional intrastomal device 122 (one example of a retaining member or a means for dispersing pressure) which is positioned within the stoma defined by the intussuscepted tissue 116. The intrastomal device 122 may serve to disperse the pressure created by the tissue anchors 120 in order to reduce pressure ulceration, which may improve retention of the separating device 118. The intrastomal device 122 may be composed of various materials, such as surgical mesh or biological skin substitutes.

FIG. 4 shows another embodiment in which the separating device 118 is positioned over the intussuscepted tissue 116, and the tissue anchors 120 traverse both an intrastomal device 122 as well as an additional extrastomal device 126 which is positioned over the intussuscepted tissue 116 within the hollow organ. The extrastomal device 126 may serve to disperse the pressure created by the tissue anchors 120 in order to reduce pressure ulceration, which may improve retention of the separating device 118. The extrastomal device 126 may be composed of various materials, such as surgical mesh or biological skin substitutes. The distal end 124 of the intussuscepted tissue 116 may, but need not, extend past one or more retaining members and/or pressure-dispersing elements, such as the intrastomal device 122 and the extrastomal device 126.

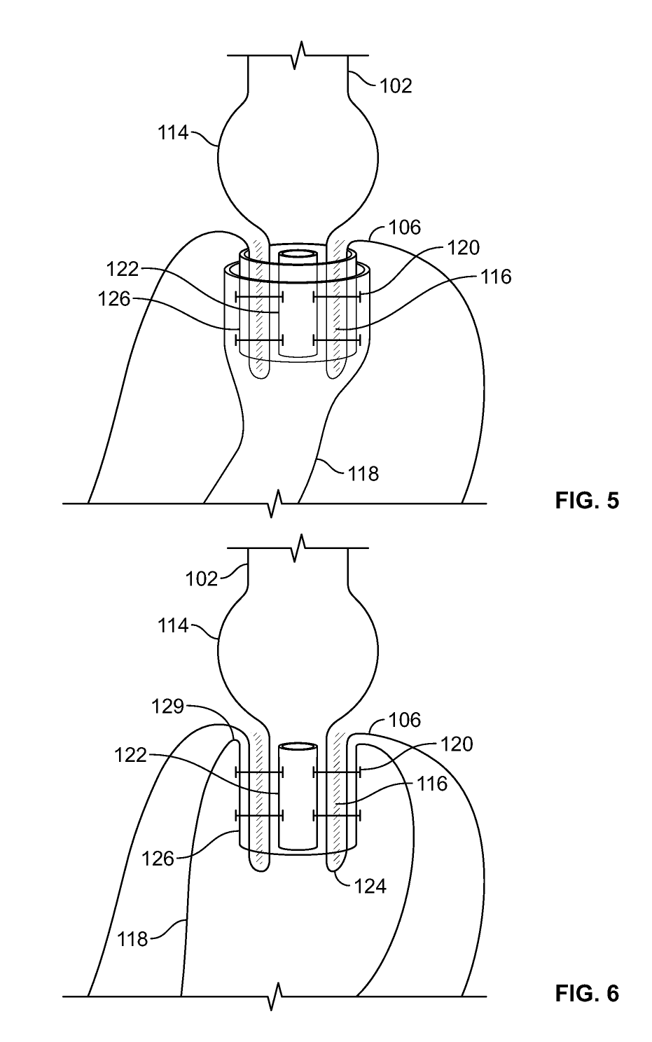

FIG. 5 shows another embodiment in which the separating device 118 is elastic and positioned over or outside of the intussuscepted tissue 116, such that the separating device is retained by its elasticity. The elasticity of the separating device 118 may create friction with the extrastomal device 126, which may serve to retain it in place. In additional embodiments, the friction can be increased by using high-friction materials for either or both the separating device 118 or the extrastomal device 126. In other embodiments, the friction can be increased by having small inward-directing barbs or hooks on the proximal end of the separating device 118 or on the extrastomal device 126 that interface with corresponding outward-directing barbs or hooks on the other component (extrastomal device 126 or separating device 118). Note in this embodiment shown in FIG. 5 that the tissue-penetrating retaining members or anchors 120 pass through the intrastomal device 122, the intussuscepted tissue 116, and the extrastomal device 126 but do not pass through the separating device.

In any of these embodiments, the separating device 118 may have various geometries. FIG. 6 shows one variation in which the tubular separating device 118 is positioned such that the proximal opening of the tubular separating device is oriented toward the distal end of the intussuscepted tissue. In this manner the separating device 118 curves up and around, creating a bend 129 as it follows the contour of the hollow organ wall, or by the force of gravity, or is prebiased, or some combination of these elements, before extending distally and may be thought of as an extension of or directly connected to an extrastomal device 126. Furthermore, also depicted, the separating device 118 may have a variable diameter along its length.

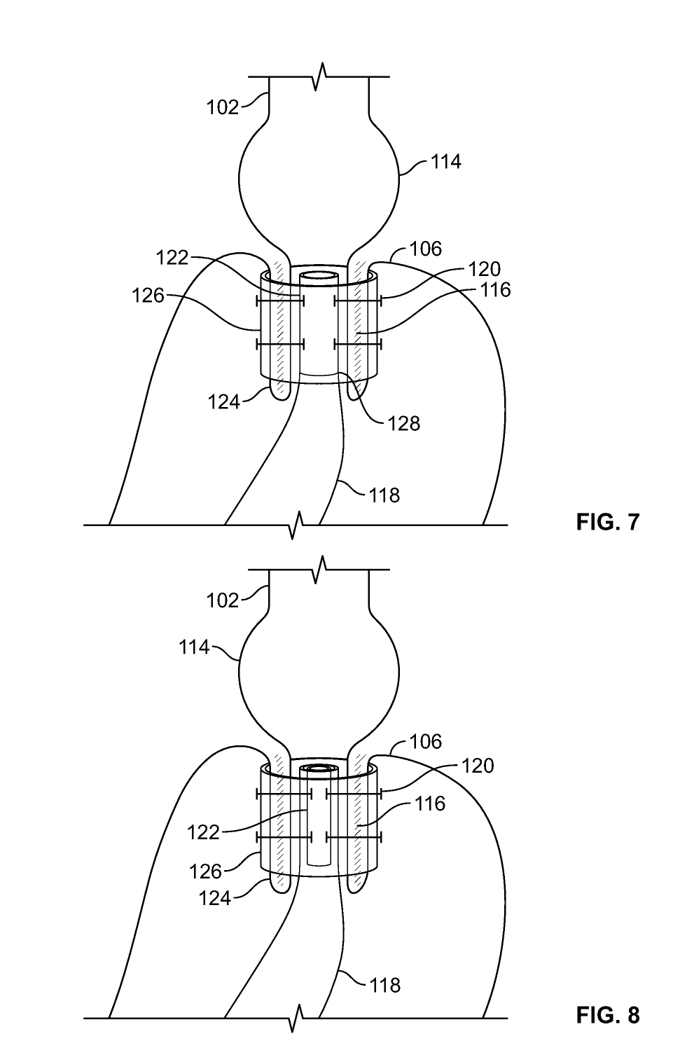

FIG. 7 shows another geometry in which the separating device 118 is positioned within the stoma defined by the intussuscepted tissue 116. The separating device 118 may be an extension 128 of or directly connect to an intrastomal device 122. In some embodiments and as shown here, tissue anchors 120 traversing the intussuscepted organ tissue as well as the extraluminal tissue may serve to anchor the separating device 118. In other embodiments, the separating device 118 has an outer diameter which fits snugly with the stoma defined by the intussuscepted tissue 116, such that friction created by this snug fit serves to retain the separating device 118 without the need for tissue anchors 120 directly anchoring or physically penetrating the separating device 118.

FIG. 8 shows another embodiment in which the separating device 118 is positioned within the stoma defined by the intussuscepted tissue 116, with a separate intrastomal device 122 rather than the separating device being an extension of the intrastomal device as in FIG. 7. The intrastomal device 122 is depicted as positioned within the separating device 118, but it may also be positioned between the separating device 118 and the intussuscepted tissue 116. The intrastomal device 122 may serve to disperse the pressure created by the tissue anchors 120 in order to reduce pressure ulceration, which may improve retention of the separating device 118. The intrastomal device 122 may be composed of various materials, such as surgical mesh or biological skin substitutes.

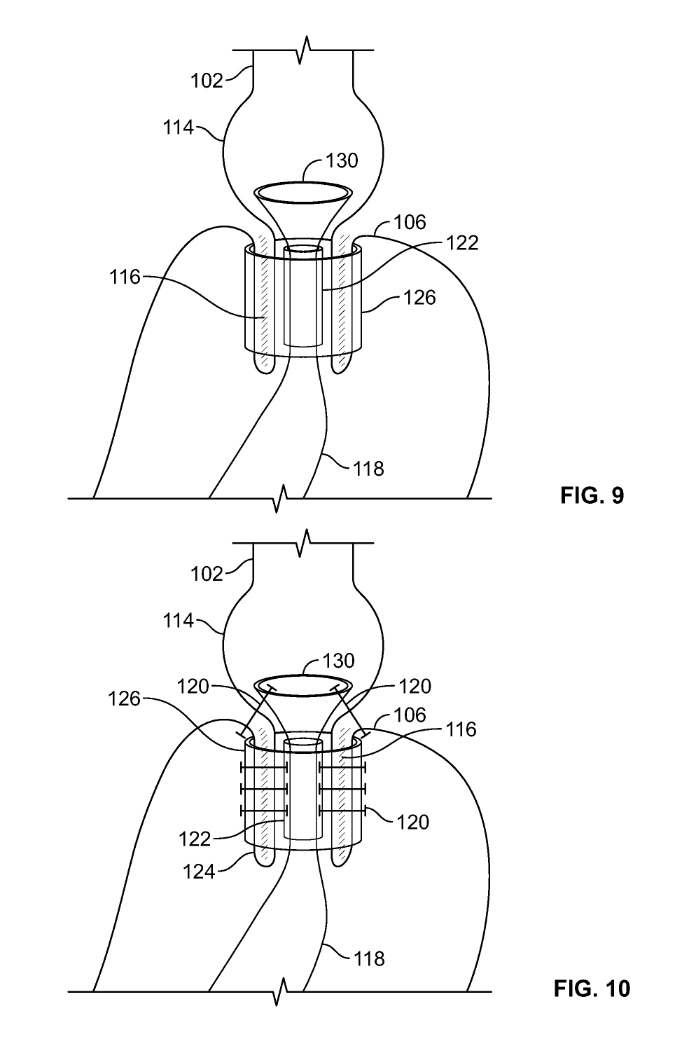

FIG. 9 shows another embodiment in which the separating device 118 has a geometry such that its proximal end 130 is too large to fit through the stoma defined by the intussuscepted tissue 116. This configuration serves to retain the separating device 118 in a region of the intussusception by trapping the separating device 118 in a proximal cavity 114 on a proximal side of a stoma formed by the intussuscepted tissue 116, thereby hanging the separating device 118 above the intussuscepted tissue 116 in a proximal cavity 114 that may be formed through an intussusception 116. The proximal end 130 of the separating device 118 is composed of a material capable of forming a geometry which resists compression, to retain this proximal end 130 within the proximal cavity 114 of the organ. For example, the proximal end may be enlarged through deployment, expansion, inflation, swelling, and the like. Supplemental or complementary mechanical tissue anchors 120 may not be necessary, but they may also be used, as depicted in FIG. 10.

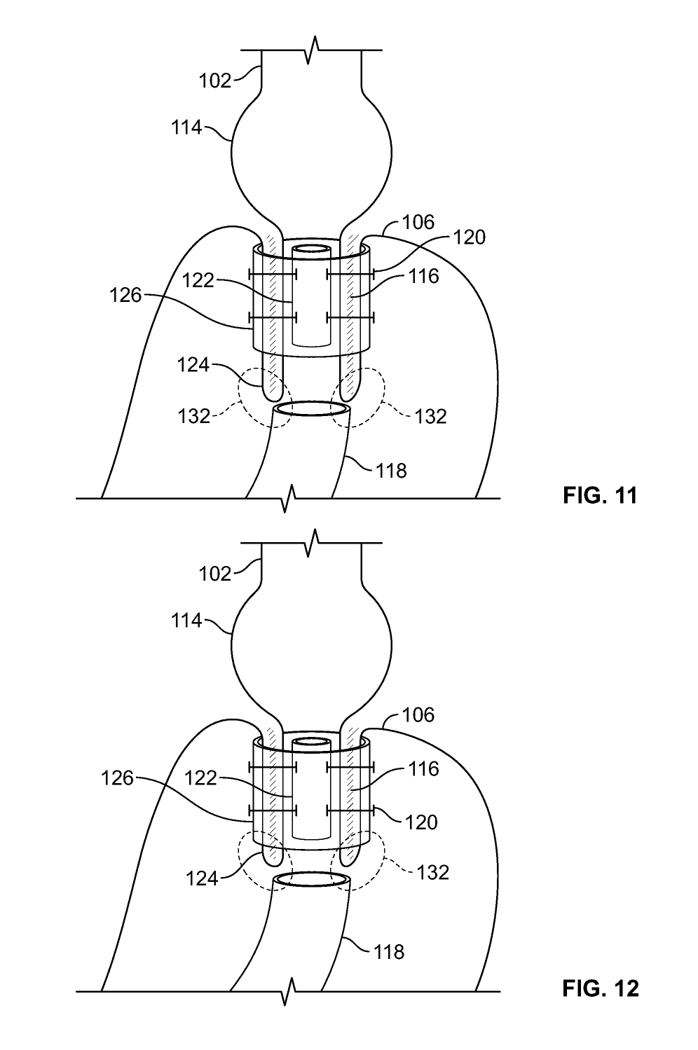

The separating device 118 need not be retained in a position outside of or within the intussuscepted tissue 116. FIG. 11 shows another embodiment in which the separating device 118 is positioned at the distal end 124 of the intussuscepted tissue 116 and retained with an intermediate connector 132. The intermediate connector 132 may be tissue anchors or rings that serve to secure the separating device 118 to the intussuscepted tissue 116, to the distal end 124 of the intussuscepted tissue, to other devices including but not limited to intrastomal devices (e.g. inner mesh, silicone bands, and the like), to extrastomal devices (e.g. outer mesh, silicone bands, and the like), to the separating device itself, to any elements connected to the intussuscepted tissue or its retaining devices, or to any combination of the aforementioned options. For example, FIG. 12 shows the intermediate connector 132 holding the separating device 118 extending through both the intussuscepted tissue 116 and the extrastomal outer mesh/band 126.

The devices described here serve to deliver and retain separating devices 118 within an intussuscepted hollow organ in the body. Also described here are devices which can remove the separating device 118 from the hollow organ after it has been previously delivered into position.

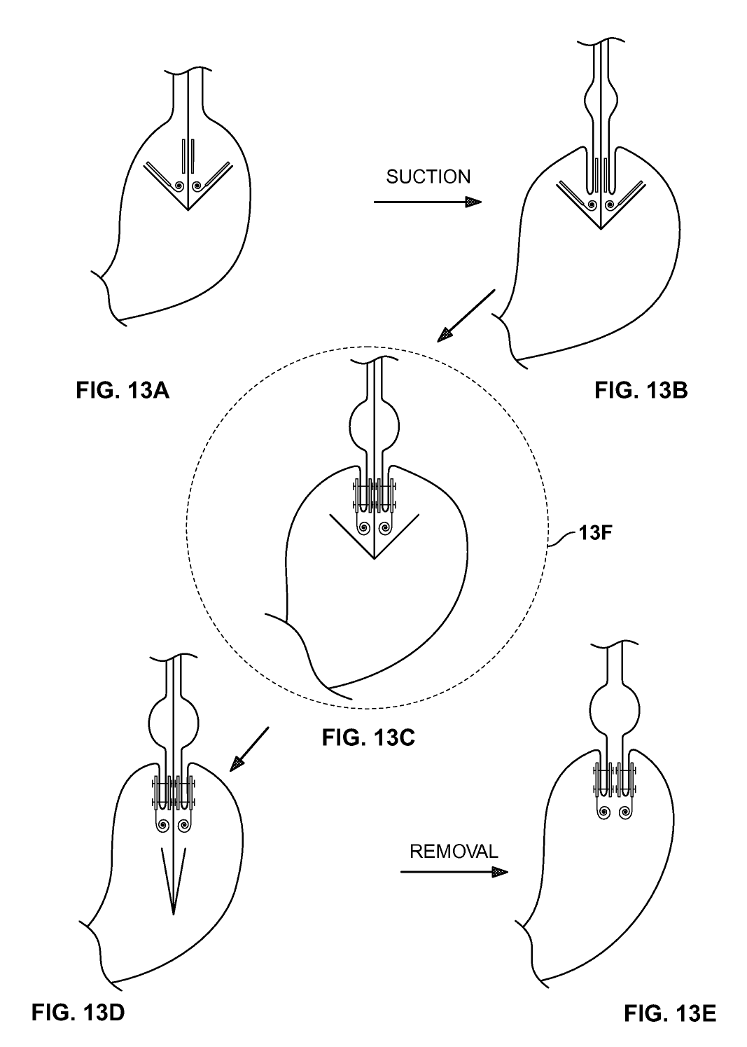

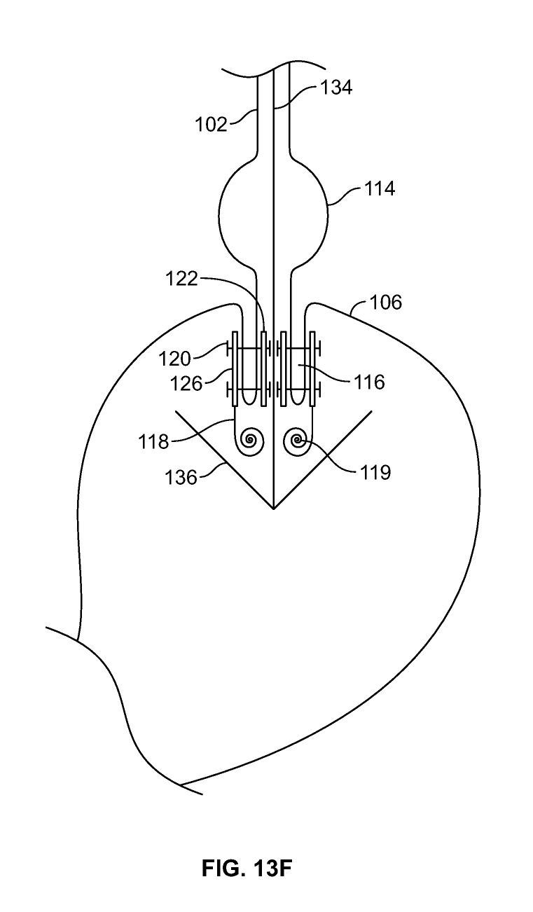

FIG. 13 shows an overview of the procedure of forming the intussusception and delivering the separating device retained to the intussusception. FIG. 13A shows a cross-sectional diagram, demonstrating a delivery device 134/136 which is inserted into the hollow organ, including both an intrastomal inner mesh/band 122, which in its final position becomes an intrastomal device, and extrastomal outer mesh/band 126, which in its final position becomes an extrastomal device. The extrastomal outer mesh/band 126 has a distal extension in a compacted configuration 119. In the embodiment shown, the compacted configuration is demonstrated by a rolled-up geometry 119. FIG. 13B shows the shape of the hollow organ once suction has been used to conform the organ to the delivery device 134/136. Alternatively, means other than suction may be used to reconfigure tissue around the delivery device 134/136 in order to form an intussusception 116. Other means might include blowing rather than suction, physical prodding with mechanical instruments, or use of magnets. Or, fine suture string could be formed along the walls of the hollow organ to form a loop and when the suture string is pulled the tissue reconfigures itself to create an intussusception 116. The intussusception 116 results when tissue is folded over upon itself around a closed loop comprising the walls of the hollow organ. If the hollow organ is rounded and tubular where the intussusception is formed then the intussusception will be circular or ovoid in shape. This configuration, along with the interserosal fibrotic tissue created by the intussusception, provides needed stability and strength to retain the separating device 118 while diffusing pressure so that no one point is stressed. Diffused pressure and managed stress maximize retention life of the separating device 118 in the hollow organ. FIG. 13C shows the hollow organ after tissue anchors 120 have been used to fasten the intrastomal inner mesh/band 122 and extrastomal outer mesh/band 126 to the double layer of intussuscepted organ wall tissue 116. FIG. 13D shows the delivery device 134/136 then being returned to a collapsed configuration just prior to removal through the channel. Delivery and removal of the device used to form the intussusception and retain the separating device to it is preferably transoral. The intrastomal inner mesh/band 122 and extrastomal outer mesh/band 126 are anchored to the intussuscepted tissue 116, with the distal extension 118/119 of the extrastomal outer mesh/band 126 as the separating device 118/119, in compacted form 119. FIG. 13E shows the intrastomal inner mesh/band 122, extrastomal outer mesh/band 126, and separating device 118 anchored to the intussuscepted tissue 116, with the delivery device 134/136 completely removed. At this point, the separating device 118/119 is still in a compacted configuration 119 and is ready to be expanded.

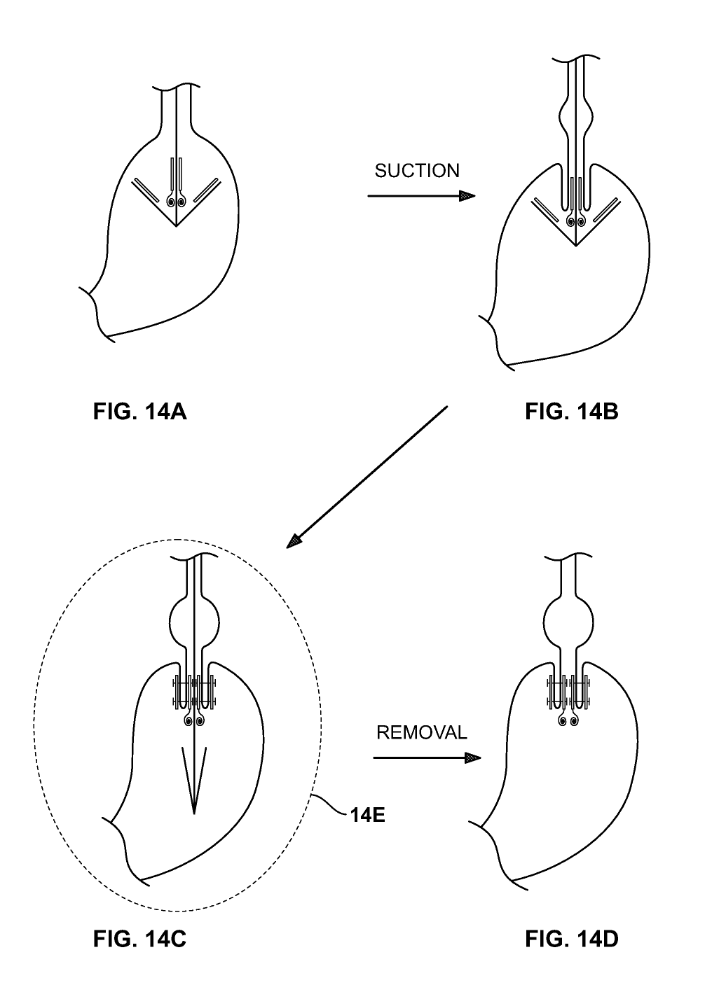

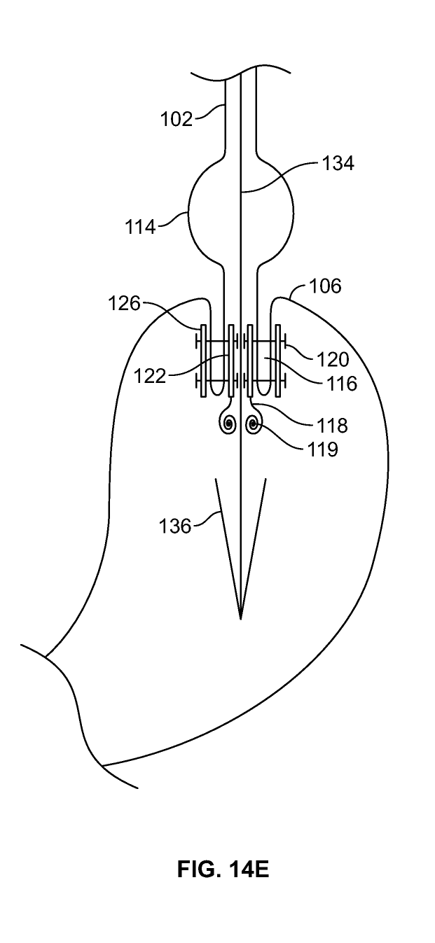

In analogous fashion, FIG. 14A-14D show a similar sequence of steps in a different embodiment, where the compacted separating device 119 is a distal extension of the intrastomal inner mesh/band 122 which in its final position becomes an intrastomal device.

In another manner of deployment, the separating device is connected to the inner mesh and is initially situated overlying the shaft of the delivery device in “inside-out” fashion, such that the most distal part of the separating device begins the procedure at the most proximal end of the device, close to the operator handle (like a sock which is flipped inside-out). The deployment mechanism is used as in typical fashion to deploy the inner and outer mesh, and secure these into place, and then the deployment mechanism is removed, leaving the separating device still in inverted fashion, now situated within the esophagus. Finally, a second device is used (either an endoscope, or other transoral device) to push the separating device distally, reversing its inverted configuration, such that the separating device is advanced out of the esophagus and into a more distal region of the GI tract.

After the separating device 118/119 is anchored in place, the separating device is expanded from its compacted or retracted configuration 119 to an expanded, extended, or deployed configuration. This can be done with any feasible means or device including using a physical unfurling element 138, fluid pressure (air pressure, a water stream), or using a biological, chemical, or physical catalyst to incite self-expansion of the separating device 118/119, etc. According to a preferred embodiment, a physical unfurling element 138 is used to deploy the separating device to its intended final position within the hollow organ.

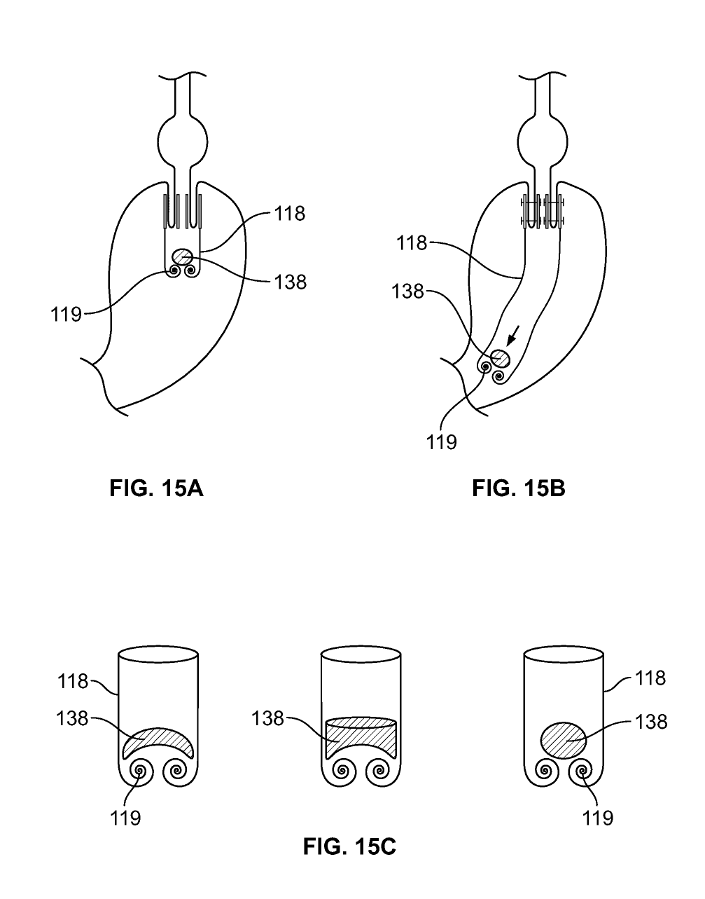

FIG. 15A shows a mechanism for expanding the separating device, where an unfurling element 138 is placed at the proximal edge of the compacted configuration 119 and then pushed distally. The distal push can be delivered using pressurized fluid or air, as shown in FIG. 15B. As the unfurling element 138 is pushed distally, it expands the separating device 118/119 into its fully expanded configuration 118. The unfurling element 138 then passes through the separating device. FIG. 15C shows various embodiments of the unfurling device 138, with the unfurling device 138 having shapes that promote its ability to expand the separating device 118/119. In these embodiments, the edges of the unfurling device 138 are positioned against the edge of the separating device 118/119, with distal edges positioned in apposition with the compacted separating device 119. For example, the unfurling element 138 may have a ball or a half-moon shape or may be cylindrical and flat at one end while curved at the other end. The unfurling element 138 may be made of various materials, including but not limited to polymers or metal. The unfurling element 138 may be designed and manufactured to have a low-friction edge, such that it glides smoothly in a distal direction as it expands the separating device. The unfurling element 138 may also be made of biologically degradable materials, including but not limited to polylactic acid (PLA), polyglycolic acid (PGA), and/or a combination of the above or related polymers.

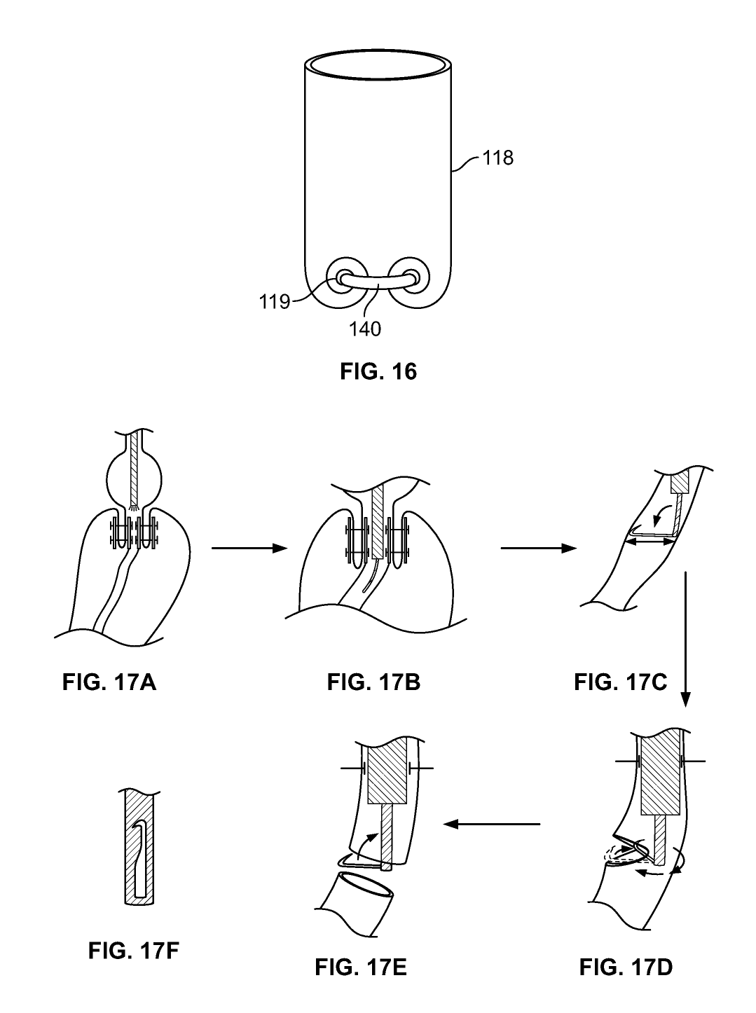

FIG. 16 shows another embodiment of the unfurling device. The unfurling device may take the shape of a complete or near-complete loop 140 positioned within the compacted separating device 119. It may be constructed such that the size of the unfurling device 140 holds the distal end of the compacted separating device 119 in sufficient apposition, such that pressurized fluid or air causes the separating device 118/119 to expand. Alternatively, the unfurling device 140 may be composed of an elastic material for this same purpose.

FIG. 17 shows an embodiment of a device which can be used to remove a previously delivered and retained separating device 118. FIG. 17A shows the previously delivered and retained separating device in a hollow organ, with the insertion of an endoscope into the organ for visualization. FIG. 17B shows the removal device inserted through the working channel of the endoscope. Alternatively, the removal device can be inserted into the hollow organ side-by-side with the endoscope and not through the working channel. FIG. 17C shows the removal device in deployed configuration, with a folding arm extended at approximately a right angle to the main shaft of the removal device. The folding arm in this embodiment has a hook-shaped end which is capable of cutting the separating device, though it need not be hook-shaped. The cutting end of the folding arm is oriented such that it will cut the separating device when the removal device is spun circumferentially or alternatively when the arm is rotated about the main axis. Furthermore, as shown in FIG. 17C, the folding arm is designed to be long enough such that when the removal device is in deployed configuration, the arm causes the separating device to have a configuration that enables the cutting end to catch the separating device for the cut. For example, in this depiction, the separating device is a soft tube, and the deployed folding arm causes the tube to assume an ovoid configuration when viewed on cross-section. This ovoid configuration makes it difficult for the folding arm to spin circumferentially without the cutting end catching on the separating device. In manners such as this, the geometry of the folding arm relative to the separating device enhances its ability to cut the separating device. FIG. 17D depicts completion of the cut by circumferentially spinning the removal device or rotating the folding arm about the axis of the main shaft. FIG. 17E shows how the folding arm is retracted into the collapsed configuration before the removal device and the endoscope are pulled out of the organ. FIG. 17F shows a frontal view of the distal end of the removal device, with the folding arm in retracted configuration. In this depiction, the hook-shaped end is shown to be oriented in a plane perpendicular to both the shaft of the removal device and the geometric plane within which the folding arm can swivel.

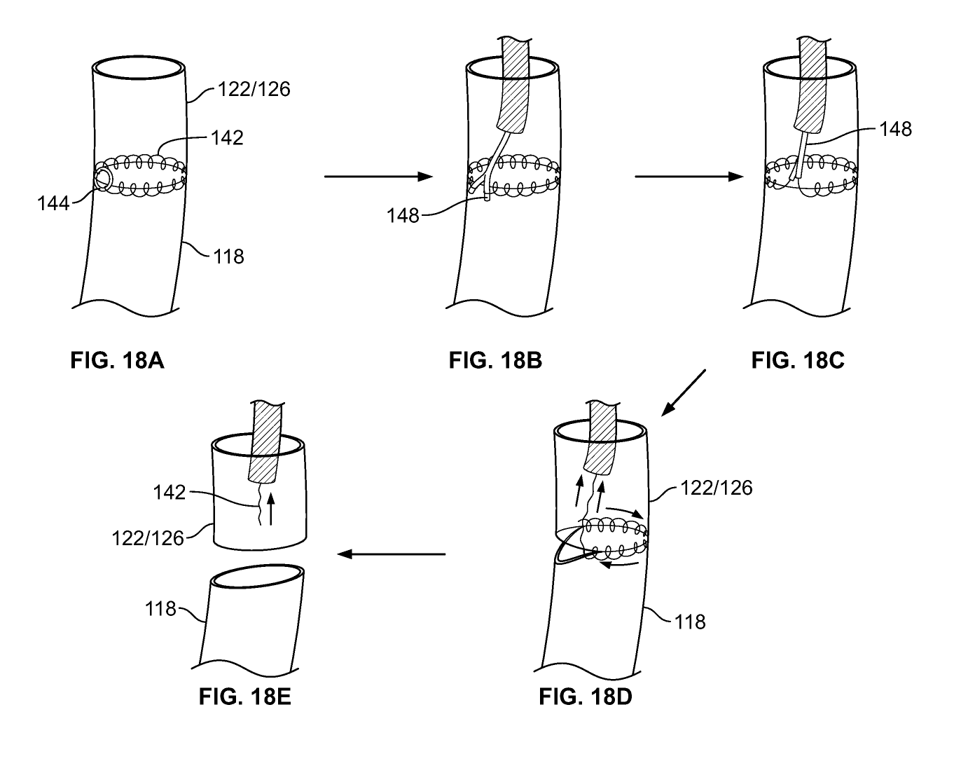

FIG. 18 shows an alternative device used to remove the anchored separating device, a specialized running suture 142. FIG. 18A shows the separating device 118 which has been secured to the intrastomal inner mesh/band 122 or extrastomal outer mesh/band 126 which in their final positions become an intrastomal device and an extrastomal device, respectively. In this embodiment, the separating device 118 has been secured to the mesh using a running suture 142. The running suture 142 is created in a manner so as to have a prominent loop 144 at one or more points along the suture 142 which facilitates cutting by endoscopic forceps 148 or other appropriate device (FIG. 18B). FIG. 18C shows endoscopic forceps 148 pulling on the cut end of the running suture 142, and FIG. 18D shows unraveling of the anchoring suture as the running suture 142 is pulled out and the forceps are retracted upward, for example through the channel of an endoscope. The separating device 118 is shown being disconnected from the mesh 122/126. FIG. 18E shows the suture completely removed, with the separating device 118 completely disconnected from the mesh 122/126. The separating device 118 can then be retrieved endoscopically and pulled out proximally.

Other embodiments utilize other, similar specialized anchoring or retention devices designed to facilitate detachment of the separating device from the intussuscepted tissue or intermediate retaining member (e.g. mesh). For example, the running suture shown in FIG. 18A can feature, instead of a prominent loop 144, a prominent connector which completes the circumferential running suture. The prominent connector resides within the lumen of the depicted tube and is designed to have some physical profile which would create resistance for an endoscopic bougie to pass through. The connector can be designed to “pop-off” such that force exerted by the bougie attempting to pass through the tube causes the connector to fall off of the suture that it is retained by, revealing free ends of the running suture 142. Removal can then proceed as seen in FIG. 18C-18E.

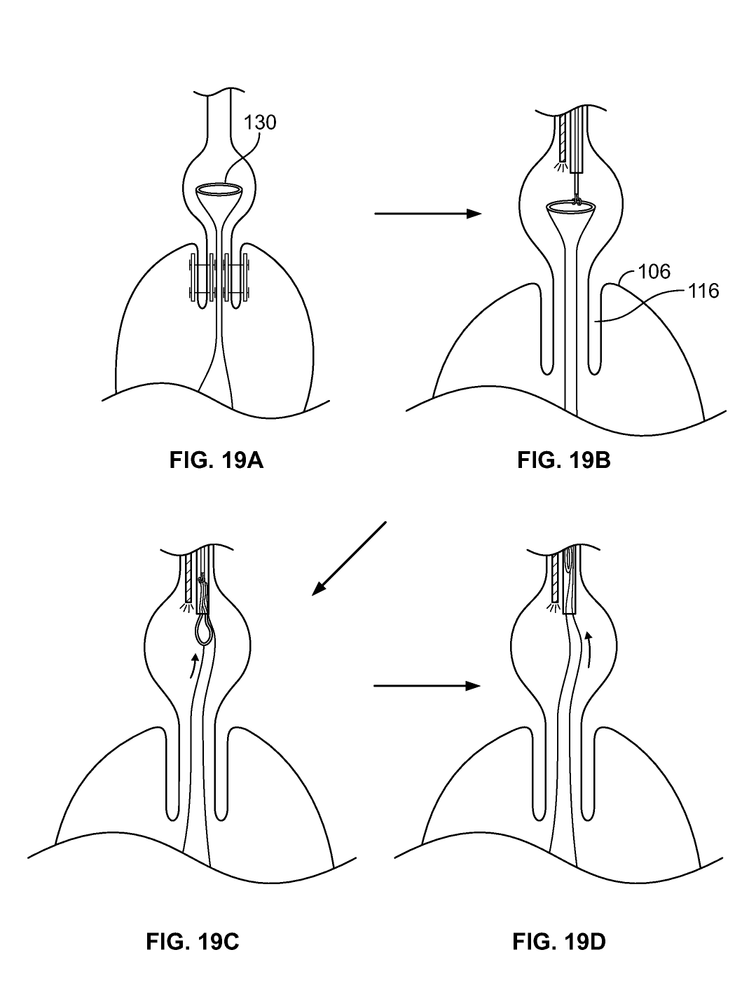

FIG. 19 shows how the design of the proximal portion 130 of the separating device 118 can facilitate removal of the device endoscopically. The retained device shown in FIG. 19A consists of a geometry, at the most proximal portion 130 of the separating device 118, which is too large to pass through the restrictive stoma. It is depicted here as a circular ring, but can also be ovoid, rectangular, or any shape which won't pass through the stoma without manipulation. It may be made of nitinol, stainless steel, polymers, or any other rigid or semi-rigid material that resists deformation into a geometry which would facilitate passage through the restrictive stoma. FIG. 19B-D show the removal of such a device. Endoscopic forceps would be used to grasp the proximal geometry and to deform it into a configuration with a smaller cross-sectional profile. FIG. 19C-19D show how the forceps could pull the proximal geometry of the separating device into a hollow tube, thus facilitating removal of the entire separating device endoscopically.

The geometry of the proximal portion 130 of the separating device 118 need not be solid. For example, it may be a balloon which is air- or fluid-filled, such that when the balloon is filled, the geometry is too large to pass through the restrictive stoma. In such a case, puncture of the balloon using endoscopic instruments will then allow the user to deform it into a geometry with a small cross-sectional profile so as to allow retrieval endoscopically.

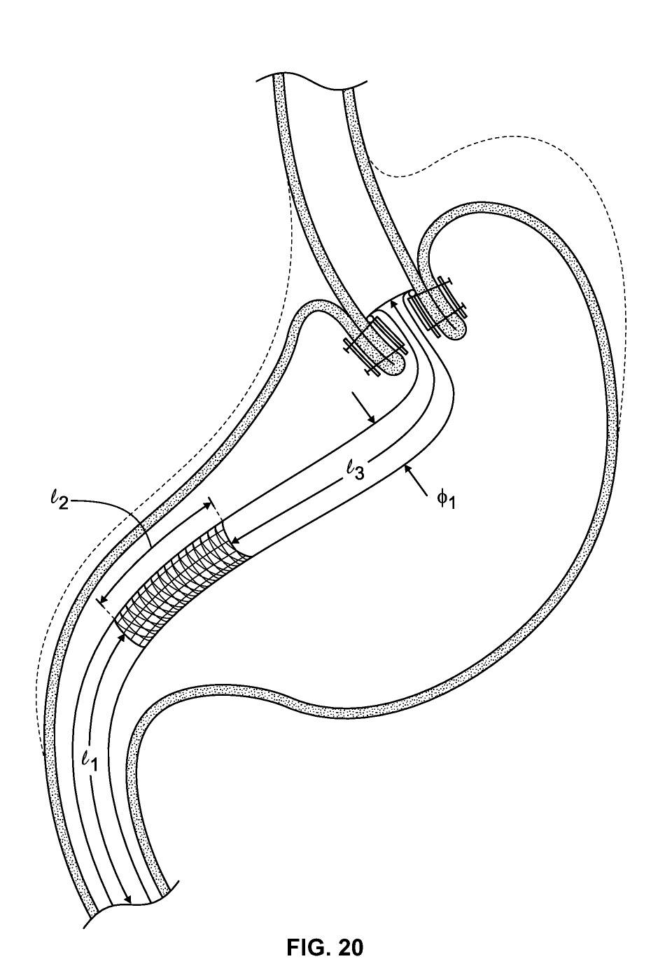

FIG. 20-22 illustrate options for the middle and distal portions of the separating device or bypass sleeve. As shown in FIG. 20, the separating device may include different segments of varying length and composition which can be adjusted depending upon the particular application and patient need. It may also be possible to interchange and adjust these segments as needed in the same patient throughout the course of their treatment. Some segments may be formed of an impermeable liner while other segments are formed of a semi-permeable or permeable fine mesh that permits partial or total passage of gastric fluid to allow partial or limited digestion. One or more segments of the separating device may be made of the following materials but is not limited to these: polypropylene (PP), polytetrafluoroethylene (PTFE), ePTE, PDMS-ePTFE, PDMS-PV, and the like.

At the proximal end of the separating device, as shown in FIG. 20, the bypass liner originates at the intussusception. As shown, at the intussusception the liner may be stabilized by a tag to the outer band. The tag extends through tissue layers from the bypass liner origin to the outer band. The bypass liner origin may be at the top or bottom of the inner band. It may be secured through tags or it may be crimped to the top or bottom of the inner band or to the bottom of the outer band.

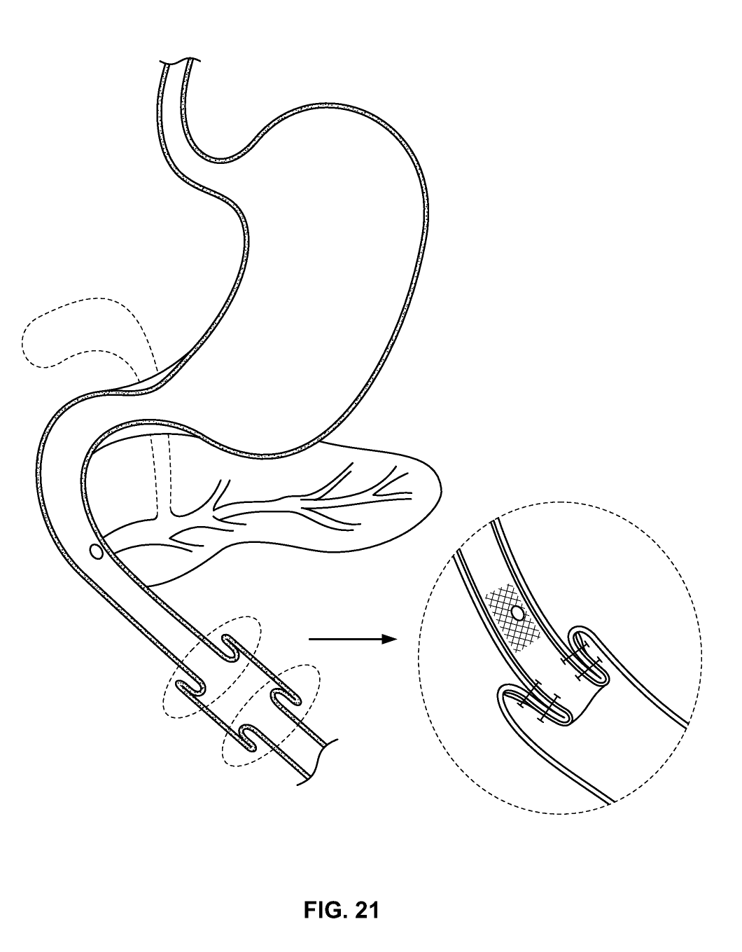

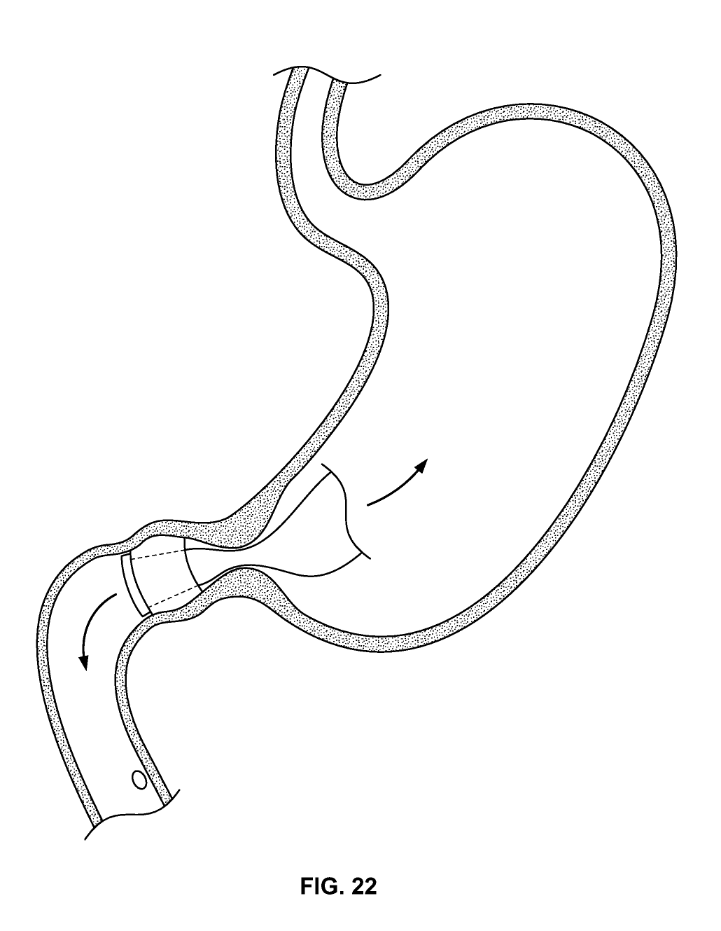

At the distal end of the separating device, it may be secured by attachment to a second distal intussusception, as shown in FIG. 21, or by a balloon, as shown in FIG. 22. As shown in FIG. 21 the distal end of the separating device is secured by intussuscepting the small intestine either above or below the outflow portal from the gallbladder or pancreas. Both configurations with an intussusception above and below the outflow are shown. However, it is likely that only one, either above or below, would be necessary to adequately secure the liner at the distal end of the separating device. The distal end of the separating device may be secured to one or more intussusceptions as shown. Also as shown in FIG. 21 a mesh may be provided in a portion or segment of the bypass liner in the vicinity of the outflow tract of the pancreas in order to provide limited exposure to digestive enzymes and bile.

FIG. 22 illustrates the distal end of the separating device secured in position in the body by a balloon. The balloon may be secured distal to the pylorus by expanding its diameter until a friction fit is created as the balloon is biased outwardly against the hollow organ walls. Outflow may be stabilized by the luminal stiffness or hoop strength achieved by material, coils, stents, or hoops/rings. The balloon may be punctured or gradually deflated if removal of the separating device is required.

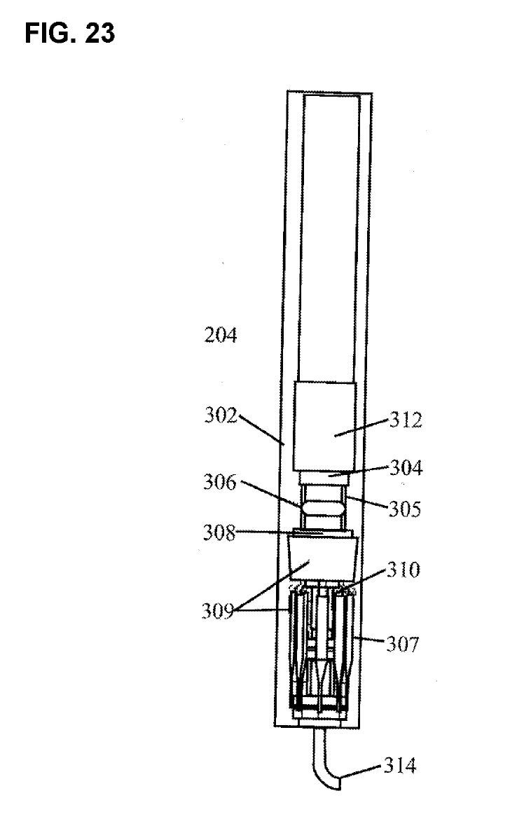

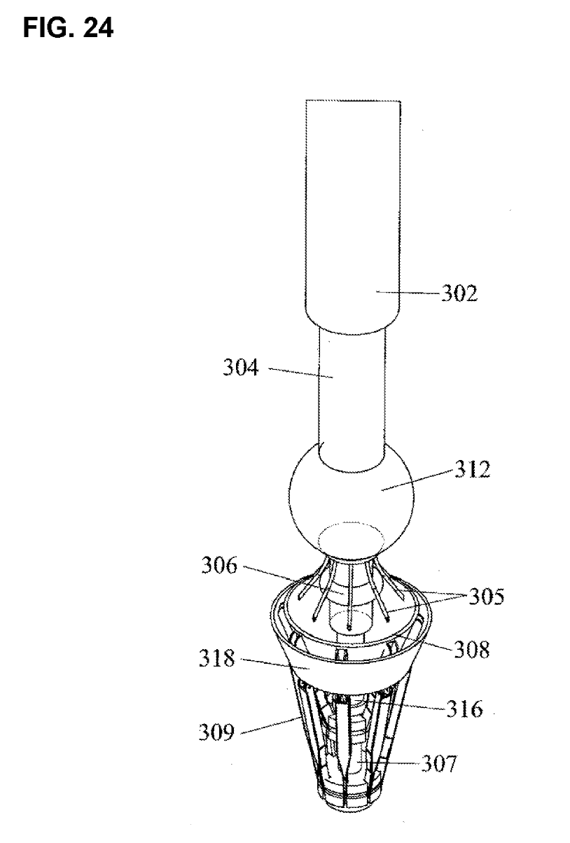

Shown in FIG. 23 is one variation of a distal portion 204 of a device for forming an intussusception, including sheath 302, holder 304, anchor introducers 305, anchor introducer expander 306, main shaft 307, retaining material 308, expandable member 309, and sizing component 312. In this variation, sheath 302 covers most of distal portion 204, and is slidable with respect to distal portion 204. In this way, the intussusception-forming device 200 may be advanced in a low profile manner to a target site of interest. The sheath may also serve to protect the individual components of the device 200 from disrupting esophageal tissue while the device 200 is advanced to a target location transorally. While shown in FIG. 23 as having a length that covers most of distal portion 204, the sheath 302 need not have such a length. Indeed, the sheath 302 may only cover a portion of distal portion 204, and in some variations, the sheath 302 only covers or partially covers expandable member 309. In other variations, the device simply does not comprise a sheath. When a sheath is used, it may be made of any suitable biocompatible material, and is most typically in the form of a flexible tube (e.g., a polymeric tube, such as one made of polyesters, polyimides, polyurethanes, combinations thereof, and the like). The sheath may also comprise one or more metals, which may be formed in any suitable fashion (e.g., braided metallic ribbons, coils, and the like). Suitable metals include, but are not limited to, stainless steel, aluminum, nickel-titanium alloys, and combinations thereof. In some variations, when the sheath 302 is withdrawn proximally, sizing component 312 and/or the expandable member 309 automatically expands. In these variations, the sizing component and/or the expandable member is made of a self-expandable material, as will be described in more detail below. The sheath 302 is shown partially withdrawn or proximally retracted in FIG. 24.

Holder 304 is configured to hold, house, couple to or with, or otherwise engage anchor introducers 305 at their proximal ends (or at their proximal portions). Holder 304 should be made of a biocompatible material, and is typically in the form of a flexible tube. The holder may be made of the same or different materials, than those of the sheath. Anchor introducers 305 may be held or otherwise attached to holder 304 in any suitable manner. For example, the anchor introducers 305 may be held in grooves formed in holder 304, the grooves having shapes corresponding to the shapes of the outer surfaces of the anchor introducers 305. The anchor introducers 305 may be snap-fit into or with the holder 304, but need not be. Indeed, the anchor introducers may simply be held in a friction-fit fashion between the grooves in the holder 304 and the main shaft 307 of the device. The anchor introducers 305 may also be attached to the holder 304 mechanically (e.g., using pins, screws, etc.), by using glue or other adhesives, or the like. The anchor introducers may also be housed within a portion of the expandable member, or a housing off the expandable member 309.

The anchor introducers 305 shown in FIG. 23 have tissue-piercing tips, but the tips need not be tissue-piercing and the tips need not be pointed. They may be blunt, or may have points with one or more beveled surfaces thereon. The anchor introducers 305 are typically made of a flexible material having a lumen capable of housing one or more anchors therein, although it should be understood that the anchor introducers need not be made of a flexible material. The anchor introducers may be made of the same or different materials, than those of the sheath. In some variations, the anchor introducers 305 are made of stainless steel hypotubes. While two anchor introducers 305 are shown in FIG. 23 and five are shown in FIG. 24, any number of anchor introducers 305 may be used (e.g., 1, 2, 3, 4, 5, 6, or more). In some variations the device comprises one anchor introducer 305. In other variations, the device comprises six or more anchor introducers 305. Also, while the anchor introducers 305 are shown in FIGS. 23 and 24 as having the same length, the anchor introducers 305 may have different lengths, and may be arranged in any suitable configuration. For example, the anchor introducers 305 may be uniformly spaced or non-uniformly spaced, and may or may not be spatially layered (e.g., the tips or ends of the anchor introducers may be closer or further from the main shaft 307).

The anchor introducers 305 are typically configured to radially expand and pierce through an intussusception, although as noted above, the anchor introducers need not be configured to pierce through tissue (e.g., may instead be used to position the anchors prior to deployment). In the variation shown in FIGS. 23 and 24, the anchor introducers 305 are also configured to pierce through at least a portion of retaining material 308, and are expanded by anchor introducer expander 306. After at least a portion of the retaining material 308 has been pierced by the anchor introducers 305, one or more anchors are deployed therethrough, as will be described in more detail below with reference to the methods. In an alternate placement (not shown) for the anchor introducers 305 they are positioned at the ends of an expandable member 309. The anchor introducer expander 306 may be any suitable component capable of aiding the radial expansion of the one or more anchor introducers 305. For example, the anchor introducer expander 306 may be a balloon (as shown in the variation of FIGS. 23 and 24), an expandable cage, one or more radially expanding prongs, or the like. The anchor introducer expander 306 may also be a pulley system, a pulling mechanism, or the like. It need not be a single component as depicted in FIGS. 23 and 24.

The retaining material 308 should be made of a material capable of retaining the stomach tissue in its intussuscepted configuration. For example, the retaining material may be made of an elastomeric material, such as biocompatible rubbers, polyurethanes, polyesters, nylons, etc.), may be made of a super-elastic or shape memory material (e.g., nickel-titanium alloys and the like), or may be made of other suitable materials. The material may be porous (e.g., mesh like, or woven in nature), or may not be. The retaining material may be continuous, or may be non-continuous in nature (e.g., made from more than one interconnected or interlocked pieces). All, or any portion of the retaining material may be coated, impregnated, or otherwise include a radiopaque or echogenic tag or marker to aid in visualization. The material may be configured for permanent placement in a stomach (e.g., be biocompatible and able to withstand stomach acids and the stomach environment generally) or be configured for temporary placement (e.g., be made of a biodegradable material). In instances where sufficient fibrosis is expected to occur, the retaining material may be configured to degrade over time, leaving a permanent fibrosed intussuscepted configuration. In some variations, the retaining material 308 is configured for permanent placement and is made of a continuous band of material as shown in FIGS. 23 and 24. The retaining material 308 may be of any suitable shape, be continuous or non-continuous, and have a uniform or non-uniform thickness. In the variation shown in FIGS. 23 and 24, retaining material 308 is positioned along at least an inner portion of expandable member 309, such that when expandable member 309 is expanded, and at a least a portion of the stomach is intussuscepted into a proximal cavity of the expandable member 309, the retaining material 308 abuts the intussuscepted tissue and retains the intussuscepted configuration when one or more anchors are placed therethrough.

The devices described here may further comprise a sizing component 312, shown in its delivery configuration in FIG. 23 and its deployed configuration in FIG. 24. The sizing component 312 helps to position the distal portion 204 of the device past the gastroesophageal junction, and also serves to ensure that there is enough stomach volume above the intussuscepted tissue. The sizing component 312 may also help facilitate the placement of the distal portion of the device relative to the stomach wall (e.g., by helping with angle positioning, etc.). In some variations, such as the variation shown in FIGS. 23 and 24, the sizing component 312 is a balloon. The sizing component 312 may also be an expandable cage, one or more radially expandable prongs, or the like, and may be manually expanded or self-expanding with the removal of the sheath 302.

Also shown in FIG. 23 is suction line 310 with suction inlet 316, and endoscope 314. The suction line 310 is configured to provide suction to stomach tissue to create the intussusception. While shown in FIG. 23 as located adjacent to main shaft 307, the suction line 310 and suction inlet 316 may be placed at any convenient location capable of making the intussusception. As shown in FIG. 24, the suction inlet 316 is positioned centrally with respect to the expandable member 308. This variation may be desirable to help ensure proper suction of the stomach tissue to create an intussusception of suitable depth while minimizing risk of obstruction. Any number of suction lines 310 and suction inlets 316 may be used. Alternatively, in variations where the endoscope 314 already has a port that enables suction, the endoscope may be positioned adjacent to the suction inlet 316 to provide a suction channel for helping to create an intussusception.

Endoscope 314 may be any suitable endoscopic device to provide for visualization during the creation and securing of the intussusception. For example, the endoscope may be a pediatric endoscope, or similar endoscope having a low profile. Other scopes or devices may also be inserted through, or alongside of, the lumen of main shaft 307, if desirable or useful.

FIG. 24 shows distal portion 204, where sheath 302 has been partially retracted, and sizing component 312, expandable member 309, and anchor introducer expander 306 are all shown in expanded or partially expanded configurations. The devices described here may also comprise a protective portion 318, which is also shown in FIG. 24. Protective portion 318 may be useful to prevent anchor introducers 305, or anchors, from penetrating too deeply into or through the stomach tissue. For example, the protective portion 318 serves to prevent the anchor introducers 305 from puncturing through to the outside of the stomach wall, where the puncturing is not associated with the securing of the intussusception (it should be understood that the anchor introducer pierces through to the outside of the stomach wall during the securing of the intussusecption, as will be discussed in more detail with reference to the methods). Protective portion 318 also prevents anchors from being deployed adjacent to a serosal layer. Protective portion 318 is shown in FIG. 24 as a continuous band of material, though it need not be. For example, the protective portion may be folded in a fan-shape so that, e.g., it may be expanded when the expandable member is fully expanded, or may be a thin flab of a metallic material, that is attached to the expandable member, or components thereof. Alternatively, the protective portion may comprise a safety mechanism in the anchor introducers or expandable member that limits deployment of anchor introducers or anchors, to a safe range. The protective portion may be made of any suitable material. For example, the protective portion may be made of one or more polymers, e.g., polystyrene, polypropylene, polyethylene (such as high-density polyethylene, ultrahigh molecular weight polyethylene, and the like), KEVLAR®, etc. Similarly, the protective portion may be made of one or more metals (e.g., stainless steel, aluminum, or the like). The protective portion may also be made of a combination of materials (e.g., a combination of one or more polymers and metals, etc.). In some variations, e.g., where a portion of the expandable member 309 serves the above functions, the protective portion 318 may not be necessary.

In accordance with an embodiment of the present invention the method of retaining a malabsorptive component of a weight reduction system to a restrictive component of a weight reduction system and the method of retaining a separating device in a region of an intussusception may begin with transorally advancing an intussusception-forming device, as shown in FIGS. 23 and 24, to a position inside the stomach. The intussusception-forming device has a proximal portion and a distal portion, with the distal portion including an expandable member and a material covering at least a portion of the expandable member. The device can be used to suction stomach tissue into the expandable member to telescope a first region of stomach tissue into a second region of stomach tissue, thereby forming an intussusception and a stomach portion proximal to the intussusception and distal to a gastroesophageal junction. One or more anchors can be deployed through the intussusception to secure the intussusception. In this manner the intussusception, serving as one example of a restrictive component of a weight reduction system, is formed or introduced into the anatomy. A malabsorptive component, e.g. a separating device, may then be introduced and attached to the restrictive component, e.g. the intussusception, thereby using the restrictive component to retain the malabsorptive component.

Weight reduction is most effective when a restrictive component of a weight reduction system is integrated with a malabsorptive component of a weight reduction system. The restrictive component of a weight reduction system may be formed in one or more hollow organs, for example by folding tissue or forming an intussusception. Additionally, or alternatively, the restrictive component may also be a space-occupying or volume-occupying object that is inserted to take up space within the hollow organ thereby reducing or restricting the remaining volume of the hollow organ that is available to fill with food. The space-occupying object may be a solid, liquid, or gas and may be fixed in form or transformable. For example, a transformable space-occupying object may be an inflatable balloon or a device that swells. The malabsorptive component of a weight reduction system may be an implantable separating device, a sleeve, or a membrane that is impermeable or selectively permeable to restrict the passage of materials across it. Additionally, or alternatively, the malabsorptive component may also be created by painting a coating on a surface of a hollow organ. Further, the malabsorptive component may also be created by modifying a surface texture, shape, and the like, of a tissue or a hollow organ to impede the ability of the tissue or organ to absorb calories.

Although the primary focus and advancement of the present invention is estimated to be improved retention of gastrointestinal implants and/or devices by incorporating the interserosal fibrotic tissue of an intussusception in hollow organs, in some embodiments also provided herein are advances in sleeve design. Although the present invention is suited for use with traditional, conventional, and contemporary sleeves it is also suited for use with the novel sleeves as taught herein.

As an alternative to a flexible, floppy sleeve capable of unintended or iatrogenic deformation or inversion, the present invention provides a semi-supported sleeve to hold its shape and position better. The semi-supported structure may be provided through springs or struts. Rather than an unsupported distal end the sleeve may alternatively have a semi-supported or supported distal end. One advantage of an at least semi-supported distal end is that it would be less likely to invert upon the portion of sleeve above it. Additionally, a distally semi-supported sleeve would make it easier for food to eventually exit the sleeve to make its way through the rest of the GI tract and exit the body. According to preferred embodiments, a semi-supported sleeve may be self-deploying along parts of it, such as at a top and a bottom. Additionally, parts of the sleeve, such as the bottom and other portions within narrower tracts of the anatomy, can deploy to friction fit inside the GI canal.

It will be apparent from the foregoing that while particular forms of the invention have been illustrated and described, various modifications can be made without departing from the spirit and scope of the invention. Accordingly, it is not intended that the invention be limited, except as by the appended claims.

We claim:

1. A method for retaining a malabsorptive component of a weight reduction system to a restrictive component of a weight reduction system, comprising:
introducing the restrictive component in a hollow organ;
introducing the malabsorptive component in the hollow organ; and
attaching the malabsorptive component to the restrictive component.

2. The method of claim 1, wherein the restrictive component is an intussusception formed by telescoping a first region of stomach tissue into a second region of stomach tissue, the malabsorptive component is a separating device, and the step of attaching results in retaining the separating device in a region of the intussusception.

3. The method of claim 2, wherein the step of attaching comprises securing a proximal end of the separating device in a region of the intussusception when the intussusception is formed.

4. The method of claim 2, wherein the step of attaching involves at least one element selected from the group consisting of:
(i) inserting mechanical elements through the intussusception, an intrastomal device, an extrastomal device, or the separating device;
(ii) enlarging a proximal portion of the separating device; and
(iii) delivering energy.

5. An apparatus for integrating a malabsorptive component and a restrictive component of a weight reduction system, comprising: a self-retaining separating device configured for attachment to an intussusception.

6. The apparatus of claim 5, wherein the separating device is deployable to self-retain by the separating device hanging from a stoma defined by the intussusception.

7. The apparatus of claim 5, wherein a proximal end of the separating device is expandable to the extent that it is larger than a stoma defined by the intussusception.

8. The apparatus of claim 5, wherein a proximal end of the separating device is elastic thereby enabling the proximal end to wrap around an outer surface of the intussusception.

9. The apparatus of claim 5, wherein a proximal end of the separating device is configured to bias outwardly.

10. The apparatus of claim 5, wherein the separating device has at least two states, an insertion/removal state and a retention state.

11. An apparatus for integrating a malabsorptive component and a restrictive component of a weight reduction system, comprising: a separating device and a retaining member configured to secure the separating device to an intussusception.

12. The apparatus of claim 11, wherein the retaining member comprises tissue-penetrating elements.

13. The apparatus of claim 11, wherein the retaining member comprises a material configured to respond to energy application from an electrosurgical or electrocautery instrument.

14. The apparatus of claim 11, further comprising means for dispersing pressure created by the retaining member.

15. The apparatus of claim 14, wherein means for dispersing pressure is at least one of: an intrastomal device and an extrastomal device.

16. The apparatus of claim 11, wherein a distal portion of the retaining member defines the separating device.

17. The apparatus of claim 16, wherein at least a portion of the retaining member is configured to deploy.

18. The apparatus of claim 16, wherein the retaining member is elastic for engagement with an outer surface of a lumen defined by walls of the intussusception thereby securing the retaining member about the lumen.

19. A separating device configured to be retained in a hollow organ, for altering absorption of at least one composition through at least one wall of at least one hollow organ, comprising:
an elongated tubular body that encloses a pathway along which at least one composition can travel until reaching a distal end of the separating device; and
means for retaining a proximal end of the separating device in a region of an intussusception.

20. The separating device of claim 19, wherein means for retaining the proximal end of the device in the region of the intussusception comprises interaction between a mucosal surface of an organ and the separating device.
